(19) United States
(12) Patent Application Publication
(10) Pub. No.: US 2011/0225661 A1
(43) Pub. Date: Sep. 15, 2011

(54) METHOD FOR TREATING AND PREVENTING RADIATION DAMAGE USING GENETICALLY MODIFIED MESENCHYMAL STEM CELLS

(75) Inventors: Weiwen Deng, Grand Rapids, MI (US); Aly S. Abdel-Mageed, Ada, MI (US); Anthony J. Senagore, Grand Rapids, MI (US)

(73) Assignee: SPECTRUM HEALTH INNOVATIONS, LLC, Grand Rapids, MI (US)

(21) Appl. No.: 12/677,001

(22) PCT Filed: Jun. 26, 2009
(86) PCT No.: PCT/US2009/048754

§ 371 Date: Aug. 23, 2010

Related U.S. Application Data
(60) Provisional application No. 61/075,763, filed on Jun. 26, 2008.

(51) Int. Cl. A01K 67/00; A61K 35/12; A61P 39/06; C12N 5/10

(52) U.S. Cl. 800/9; 424/93.21; 435/325

(57) ABSTRACT

A method of treating or preventing radiation damage by administering to a patient in need of treatment at least one therapeutically effective amount of a mesenchymal stem cell genetically altered to secrete extracellular superoxide dismutase is provided. Also provided is a therapeutic for treating and/or preventing radiation related or damage by similar agents, the therapeutic contains genetically modified mesenchymal stem cells capable of secreting extracellular superoxide dismutase.

### BACKGROUND OF THE INVENTION

The present invention relates to radiation damage, and more particularly, to treatments of radiation damage.

The risk to civilians, police, and military personnel of being exposed to lethal doses of ionizing radiation is greater now than ever before due to a growing possibility of a nuclear terrorism event (Zenk, Expert Opin Investig Drugs. 16: 767-70 [2007]). A typical scenario assumes that suicide terrorists would bring a ¹³⁷Cs source into a major subway station of a metropolitan city, un-shield the source, and attempt to expose as many people as possible to lethal γ radiation. As a result, several thousand or more people could be irradiated to varying dosages of whole body radiation and all would be at risk for developing some degree of acute radiation syndrome (ARS) (Fliedner, Curr Opin Hematol. 13: 436-44 [2006]). A significant number of the victims are believed to die of ARS within 60 days after receiving 4-6 Gy whole body exposure (sooner with higher doses). Clinical observation has confirmed that peripheral blood lymphocyte count drops rapidly in a dose-dependent manner within 1-2 days after total body irradiation (TBI) exposure (Fliedner, Curr Opin Hematol. 13: 436-44 [2006]; Greenberger, Pharmacogenomics 7: 1141-5 [2006]).

ARS is a condition caused by a brief whole body exposure to more than one sievert (Sv) dose equivalent of radiation. ARS is initially characterized by anorexia, nausea, and vomiting but can progress to hematological, gastrointestinal, neurological, pulmonary, and other major organ dysfunction. The degree of symptom severity of ARS is directly correlated to the absorbed dose of radiation. The cells of the body that are most vulnerable to damage from radiation are the rapidly dividing cells of bone marrow and intestinal lining (Mettler and Voelz, N Engl J Med. 346: 1554-61 [2002]; Zenk, Expert Opin Investig Drugs. 16: 767-70 [2007]). At present, no approved drugs or therapies are available for the prevention or treatment of ARS, despite the critical nature of this national security threat (Weisdorf et al., Biol Blood Marrow Transplant 12: 672-82 [2006]; Zenk, Expert Opin Investig Drugs. 16: 767-70 [2007]; Chao, Exp Hematol. 35(4 Suppl 1): 24-7 [2007]; Greenberger, Gene Ther. 15: 100-8 [2008]).

Radiation exposure also produces delayed radiation effects such as lifespan shortening, cataractogenesis, and carcinogenesis, which can occur months to decades later (Epperly et al., Radiat Res. 170: 437-43 [2008]). Further, cataract of the eyes and cancer development are the known late-effects of exposure to space radiation in astronauts, military aviators, and flight crews (Parker, Sci. Am. 294: 40-7 [2006]).

The formation of reactive oxygen species (ROS) such as superoxide anion (O₂⁻) following irradiation is a major determinant of lethality following fatal whole body radiation exposure (Zwacka et al., Hum Gene Ther. 9: 1381-6 [1998]; Mitchell et al., Ann N.Y. Acad Sci. 899: 28-43 [2000]; Murray and McEwan, Cancer Biotherapy & Radiopharmaceuticals 22: 1-23 [2007]). Radiation exposure induces oxidative damage to bone marrow and gastrointestinal tract which is the major factor causing bone marrow failure and gastrointestinal damage (Greenberger, Pharmacogenomics 7: 1141-5 [2006]; Zenk, Expert Opin Investig Drugs. 16: 767-70 [2007]). Radiation dosage is expressed in gray (Gy). At a dose of <1 Gy, the damage to cells is not severe and almost all victims survive. At a dose of 1-8 Gy, there is damage to bone marrow stem cells, resulting in hematopoietic dysfunction manifesting as decreased numbers of white blood cells and platelets, which lead to an increased susceptibility to infection and bleeding. At a dose of 8-30 Gy, there is serious damage to the gastrointestinal tract. The absorbed dose of radiation at which 50% of exposed individuals will die without medical support is estimated to be 3.25 Gy (Waselenko et al., Ann Intern Med. 140: 1037-52 [2004]; Zenk, Expert Opin Investig Drugs. 16: 767-70 [2007]). It is observed that irradiated tissues and organs release ROS for days to months after ionizing radiation exposure (Greenberger and Epperly, In: Progress in Gene Therapy. Columbus Frank (Ed.), Nova Science Publications, N.Y., USA, p 110-8 [2005]).

A radioprotective agent functions to protect critical body tissues against low to moderate doses of ionizing radiation and the in situ generated free radicals associated with biological tissues being exposed to such radiation. Radioprotective agents are beneficially administered to patients receiving radioisotope and radiation treatments, as well as to protect individuals entering radiation-contaminated environments. Such radioprotective agents serve antimutagenic and anticarcinogenic roles within tissues containing such agents.

The development of radioprotective agents has been the subject of intense research in view of their potential use in a radiation environment, such as space exploration, radiotherapy, and even nuclear war, for many decades. However, no ideal, safe synthetic radioprotectors are available to date, so the search for alternative sources, including plants, continues. Presently available methods and compositions for treating radiation damage require the administration of high doses of agents such as pharmaceuticals or other chemical additives by parenteral routes within a short time frame before or after the radiation or chemical insult (See e.g., Bump and Malaker, (eds.), Radioprotectors: Chemical, Biological, and Clinical Perspectives, CRC Press, Washington, D.C. [1997]). Therefore, this precludes their use as a long-term prophylactic measure for use in protection against unanticipated radiation injury.

Additionally, most radioprotective agents only have a short duration of action. Many active agents lose viability over time and may not exhibit good bulking activity or good film forming characteristics. Many active agents are insoluble in water, and thus the active agents have to be applied as aqueous emulsions. For instance, proteins and peptides may be desirable active agents, particularly for protein-based applications, but incorporation into formulations may be problematic due to their generally high levels of hydrophobicity, and incorporation into material substrates may subject them to laundering or other cleaning effects, causing loss of the active agent as well as functional efficacy, over time. This limits the potential feasibility of using such agents.

More specifically, radioprotective agents reduce the biological effects of radiation. They may be administrated before and/or after radiation exposure and can protect the organism from radiation-induced lethality. Radioprotectors have been shown to operate by a variety of different mechanisms (for review, see e.g., Bump and Malaker (eds.), Radioprotectors: Chemical, Biological, and Clinical Perspectives, CRC Press, Washington, D.C. [1997]). The mechanisms of protection can be based on the radioprotector's antioxidant properties (Weiss and Landauer, Ann. N.Y. Acad. Sci., 899:44-60 [2000]), estrogenic activity (Miernicki et al., Soc. Neurosci. Abstr., 16:1054 [1990]; and Patt et al., Amer. J. Physiol., 159:269-280 [1949]), and/or in some cases, the ability to inhibit protein kinase(s) involved in signal transduction (Liu et al., Oncogene, 19: 571-579[2000]).

Ionizing radiation increases formation of O₂⁻ which causes DNA strand breaks, oxidizes membrane lipids, and reacts with nitric oxide to form the toxic peroxynitrite. All of these oxidative damage processes can lead to cell apoptosis (Kanai Am J Physiol Renal Physiol. 283: F1304-12 [2002]; Jung et al, Circ Res. 93: 622-9 [2003]; Greenberger and Epperly, In: Progress in Gene Therapy. Columbus Frank (Ed.), Nova Science Publications, N.Y., USA, p 110-8 [2005]; Rodemann and Blaese, Semin Radiat Oncol. 17: 81-8 [2007]). Therefore, strategies and compositions to decrease O₂⁻ have been searched for in order to alleviate radiation-induced oxidative damage.

For example, superoxide dismutase (SOD), an antioxidant enzyme catalyzing the dismutation of O₂⁻ alleviates oxidative damage. Three SOD isoforms have been identified: cytosolic copper-zinc SOD (CuZnSOD), mitochondrial manganese SOD (MnSOD), and extracellular SOD (ECSOD). ECSOD gene therapy has recently been shown to be effective for a variety of diseases involving oxidative damage (Bivalacqua et al., Am J Physiol Heart Circ Physiol. 284: H1408-21 [2003]; Bivalacqua et al., J Sex Med. 2: 187-97 [2005]; Brown et al., Am J Physiol Heart Circ Physiol. 290: H2600-5 [2006]; Heistad, Arterioscler Thromb Vasc Biol. 26: 689-95 [2006]), but has not been used to treating radiation related damage.

Since the function of MnSOD is to remove O₂⁻ produced in mitochondria, augmentation of MnSOD expression before irradiation is thought to inhibit radiation-induced cell apoptosis. It has been demonstrated that gene transfer of MnSOD protects against radiation-induced tissue damage in mice when administrated prior to irradiation. The mechanism was considered to be a stabilization of mitochondrial membrane through the removal of O₂⁻ produced in mitochondria by MnSOD (Fridovich, Annu Rev Biochem. 64: 97-112 [1995]; Kanai et al., Am J Physiol Renal Physiol. 283: F1304-12 [2002]; Epperly et al., Radiat Res. 157: 568-77 [2002]; Epperly et al., Radiat Res. 160: 568-78 [2003]; Heistad, Arterioscler Thromb Vasc Biol. 26: 689-95 [2006]). MnSOD gene therapy studies show that overexpression of MnSOD prior to radiation exposure can provide radioprotection to normal tissues in irradiated animals (Epperly et al., Int J Radiat Oncol Biol Phys. 43: 169-81 [1999]; Kanai et al., Am J Physiol Renal Physiol. 283: F1304-12 [2002]; Epperly et at., Mil Med. 167(2 Suppl): 71-3 [2002]; Niu et al., In Vivo. 19: 965-74 [2005]). In one study, intratracheal injection of adenovirus containing human MnSOD for the overexpression of MnSOD in the lungs of mice prior to irradiation prevents mice from irradiation-induced organizing alveolitis (Epperly et al., Int J Radiat Oncol Biol Phys. 43: 169-81 [1999]). In another study, MnSOD plasmid was administrated to mice 24 hours prior to esophageal irradiation and it protected esophageal progenitors of squamous epithelium (Niu et al., In Vivo. 19: 965-74 [2005]). However, MnSOD radioprotective gene therapy has no therapeutic effect when MnSOD gene construct is administrated after radiation exposure (Greenberger et al., Curr Gene Ther. 3: 183-95 [2003]; Greenberger, Pharmacogenomics 7: 1141-5 [2006]; Greenberger, Gene Ther. 15: 100-8 [2008]).

ECSOD is a secretory Cu and Zn-containing tetrameric glycoprotein. ECSOD is the only SOD isoform that is released from cells. ECSOD is produced and secreted only by macrophages, smooth muscle cells, fibroblasts, and glia cells. It exists primarily in the interstitial space of tissues, plasma, and lymph (Marklund, Biochem J. 266: 213-9 [1990]; Stralin and Marklund, Biochem J. 298 (Pt 2): 347-52 [1994]; Choung et al., Exp Dermatol. 13: 691-9 [2004]). ECSOD is suggested to be a major determinant of nitric oxide bioavailability for the maintenance of vascular function (Jung et al., Circ Res. 93: 622-9 [2003]; Faraci and Didion, Arterioscler Thromb Vasc Biol. 24: 1367-73 [2004]). As ECSOD is found in the extracellular matrix of tissues, it is ideally situated to prevent cell and tissue damage initiated by extracellularly produced O₂⁻ (Fattman et al., Free Radic Biol Med. 35: 236-56 [2003]). Animal studies have demonstrated that adenovirus mediated ECSOD gene therapy is effective in treating a variety of cardiovascular diseases (Fennell et al., Gene Ther. 9: 110-7 [2002]; Chu, Methods Mol Med. 108: 351-61 [2005]; Bivalacqua et al., J Sex Med. 2: 187-97 [2005]; Brown et al., Am J Physiol Heart Circ Physiol 290: H2600-5 [2006]; Heistad, Arterioscler Thromb Vasc Biol. 26: 689-95 [2006]). It is also demonstrated that overexpression of ECSOD reduces acute radiation induced lung toxicity (Kang et al., Int J Radiat Oncol Biol Phys. 57: 1056-66 [2003]; Rabbani et al., BMC Cancer. 5: 1-13 [2005]). However, no effective therapy has been created to date containing ECSOD.

Despite all of the above information, there remains no good treatment of radiation damage and no animal model to study the effects both of radiation damage and the proposed treatments in more detail.

### SUMMARY OF THE INVENTION

The current embodiment provides a method for treating and preventing radiation damage by administering an effective amount of mesenchymal stem cells (MSCs) genetically modified to secrete extracellular superoxide dismutase (ECSOD). The genetically modified MSCs are known as ECSOD-MSCs.

In another embodiment, the ECSOD-MSCs are administered in a medically acceptable manner. Examples of such acceptable manners include, but are not limited to, intravenous administration, intra-bone marrow administration, intra-arterial administration, intra-cardiac injection, intracerebral injection, intraspinal injection, intrathecal, intra-peritoneal injection, intra-muscular injection, subcutaneous injection, parenteral administration, intra-rectal administration, intra-tracheal injection, intra-nasal administration, intradermal injection, oral, and combinations thereof.

In yet another embodiment, the ECSOD-MSCs can be administered to a patient at a variety of locations including, but not limited to, systemically, at the site of injury, at an adjacent site to the site of injury, and at a site remote from the site of injury, wherein the mesenchymal stem cells migrate to the site of injury after administration.

In another embodiment, the ECSOD-MSCs are administered in multiple therapeutically effective amounts. The repeated administration provides additional production of ECSOD at the location of treatment.

In another further embodiment, a therapeutic for treating and/or preventing radiation related damage is provided. The therapeutic is formed of genetically modified mesenchymal stem cells capable of secreting extracellular superoxide dismutase, known as ECSOD-MSCs. The therapeutic can be used in the method described above for treating and preventing radiation damage.

The ECSOD-MSCs are genetically modified using a vehicle to transfer cDNA of extracellular superoxide dismutase into the mesenchymal stem cells for production and secretion of extracellular superoxide dismutase. Examples of transfection vehicles include, but are not limited to, an adenoviral vector, a retroviral vector, a lentiviral vector, and a plasmid.

The MSCs for use in the therapeutic can be isolated from locations selected from bone marrow, umbilical cord blood, adipose tissue, skin, peripheral blood, and other appropriate tissues. Additionally, the MSCs can be autologous, allogeneic, syngeneic, and xenogeneic MSCs, with respect to a patient receiving the therapeutic.

The therapeutic can be used to treat or prevent radiation damage. Examples of radiation damage include, but are not limited to, cell injury, tissue damage, organ dysfunction, acute radiation syndrome, and delayed radiation effects such as radiation-induced lifespan shortening, cataractogenesis, and carcinogenesis.

In another embodiment the ECSOD-MSC therapeutic can be combined with another unrelated therapeutic, or used in combination with other treatments or therapeutics.

Yet another embodiment provides a mesenchymal stem cell genetically altered to secrete extracellular superoxide dismutase for use in therapy

A further embodiment provides the ECSOD-MSC for use in treating or preventing radiation damage or other agents having similar mechanisms of action.

Another embodiment provides the use of a mesenchymal stem cell genetically altered to secrete extracellular superoxide dismutase in the preparation of a medicament for treating or preventing radiation damage or other agents having similar mechanisms of action.

These and other objects, advantages, and features of the invention will be more fully understood and appreciated by reference to the description of the current embodiment and the drawings.

### BRIEF DESCRIPTION OF THE DRAWINGS

FIGS. 1A and 1B demonstrate the efficacy of adenoviral gene transfer in mouse mesenchymal stem cells (mMSCs). FIG. 1A is a graph depicting secretion of biologically active extracellular superoxide dismutase (ECSOD) by Ad5CMVECSOD-transduced mMSCs. FIG. 1B is a set of images depicting expression of nuclear targeted β-galactosidase by Ad5CMVntlacZ-transduced mMSCs.

FIGS. 2A and 2B demonstrate the efficacy of adenoviral gene transfer in human mesenchymal stem cells (hMSCs). FIG. 2A is a graph depicting secretion of biologically active ECSOD by Ad5CMVECSOD-transduced hMSCs. FIG. 2B is a set of images depicting expression of nuclear targeted β-galactosidase by Ad5CMVntlacZ-transduced hMSCs.

FIG. 3 shows the phenotype of hMSCs. Flow cytometric analysis was conducted on ex vivo-expanded hMSCs to determine the expression of CD14, CD29, CD34, CD44, CD45, CD73, CD90 (Thy-1), CD105, human lineage cocktail (Lin1 i.e. CD3, CD14, CD16, CD19, CD20, and CD56), and HLA-DR.

FIG. 4 is a Kaplan-Meier survival curve demonstrating that intravenous treatment with ECSOD gene-modified mMSCs improves survival of irradiated mice.

FIGS. 5A and 5B show a set of images depicting the removal of cell clumps in mouse mesenchymal stem cell (mMSC) suspension by filtration method. FIG. 5A is a photomicrograph showing mMSCs before filtration. FIG. 5B is a photomicrograph showing mMSCs after filtration through a 40 μm nylon mesh.

FIG. 6 is a graph depicting the effect of ¹³⁷Cs γ radiation on cell counts of peripheral blood in mice.

FIGS. 7A-D show the improvement in survival of irradiated mice by mesenchymal stem cells genetically modified with extracellular superoxide dismutase. FIG. 7A is a flow cytometric analysis of the phenotype of mMSCs; FIG. 7B is a graph showing the ECSOD secretion by Ad5CMVECSOD-transduced mMSCs; FIG. 7C are photomicrographs showing expression of nuclear-targeted β-galactosidase by Ad5CMVntlacZ-transduced mMSCs; and FIG. 7D is a graph showing intravenous administration of ECSOD gene-modified mMSCs improves survival of irradiated mice.

FIGS. 8A and 8B show the persistence of adenoviral-mediated transgene expression in vitro; FIG. 8A are graphs showing secretion of biologically active ECSOD by Ad5CMVECSOD-transduced mMSCs at days 0 and 35 after adenoviral transduction. FIG. 8B are photomicrographs showing expression of nuclear-targeted β-galactosidase by Ad5CMVntlacZ-transduced mMSCs at day 0 and 35 after adenoviral transduction.

FIG. 9 is a graph showing the effect of intravenous administration of ECSOD-mMSCs on body weight loss in irradiated mice.

FIGS. 10A and 10B are graphs showing the effects of irradiation doses on body weight loss and survival in mice.

FIGS. 11A-C show radioprotective effect of mesenchymal stem cells genetically modified to secrete extracellular superoxide dismutase. FIG. 11A is a Kaplan-Meier survival curve showing extended lifespan in irradiated mice intravenously treated with ECSOD-mMSCs. FIG. 11B is a set of images showing delay in cataract formation in irradiated mice intravenously treated with ECSOD-mMSCs. FIG. 11C is photograph showing prevention of carcinogenesis in irradiated mice intravenously treated with ECSOD-mMSCs.

### DESCRIPTION OF THE CURRENT EMBODIMENT

In the current embodiment, a method for treating radiation damage using mesenchymal stem cells (MSCs) genetically modified with extracellular superoxide dismutase (ECSOD), known as ECSOD-MSCs, is provided. Also provided is a method of transducing mouse or human MSCs with a vector carrying human ECSOD gene to create MSCs that produce and secrete exogenous ECSOD. Further provided is a method of intravenous MSC-based ECSOD gene therapy to improve survival of the treated organism after radiation exposure.

The term “radiation damage” as used herein is intended to include, but is not limited to, cell injury, tissue damage, tissue dysfunction, acute radiation syndrome, delayed radiation effects such as radiation-induced lifespan shortening, cataractogenesis, and carcinogenesis, and other like damage relating to or caused from exposure to radiation, as well as damage caused by other substances but which has a similar effect on cells and tissues as radiation damage. The radiation exposure can be a consequence of a number of problems including, but not limited to, a radiation accident, nuclear accident, nuclear terrorism, nuclear war, other radiological emergencies, space travel, radiation therapy, as well as diagnostic radiology. Space travel can include, but is not limited to, exposure to space radiation in astronauts, military aviators, and flight crews. Radiation-induced cataract and cancer become a major health risk in long-duration space flights such as missions to the International Space Station, the moon and Mars. Radiation therapy can include a radiation treatment of cancers including, but not limited to, leukemia, lymphoma, brain tumor, thyroid tumor, lung cancer, liver cancer, breast cancer, cervical cancer, ovarian cancer, prostate cancer, endometrial cancer, bladder cancer, colorectal cancer, and other similar cancers or diseases. Diagnostic radiology can include, but is not limited to, X-ray radiographing, CT scanning, and nuclear medicine imaging.

The term “ECSOD” as used herein is intended to include, but is not limited to, ECSOD of recombinant origin. This form is easily available in large quantities, but ECSOD is also available from other sources. Thus, the ECSOD can also be of cell line origin, i.e. derived from a cell line producing the protein in significant quantities, such as a cell line derived from blood or lung, blood vessel, pancreas, uterus, prostate gland, placenta or umbilical cord tissue, and neoplastic tissue. Endothelial cells or fibroblasts can also be sources of ECSOD. ECSOD is an antioxidant enzyme catalyzing the dismutation of superoxide anion. The ECSOD can be human extracellular superoxide dismutase or a mammalian extracellular superoxide dismutase, or of other similar sources.

The ECSOD can also be derived from tissue found to be relatively rich in ECSOD. Accordingly, the current embodiment further relates to ECSOD of placenta or umbilical cord origin as these tissues have been found to contain reasonably large amounts of ECSOD compared to other types of tissue, and are also more easily available than, for instance, lung, uterus or pancreas tissue. Although these tissues contain relatively larger amounts of ECSOD, these amounts are far smaller than those obtainable by recombinant DNA techniques, and therefore, placenta or umbilical cord ECSOD is particularly indicated for special purposes requiring only minor amounts of ECSOD.

As used herein, the term “patient” or “subject” refers to a warm-blooded animal such as a mouse, rat, cat, dog, cow, horse, monkey, and human.

The term “mesenchymal stem cells (“MSCs”)” as used herein is intended to include, but is not limited to, multipotent cells that can differentiate into a variety of cell types. Mesenchymal stem cells (MSCs), also known as marrow stromal cells, are a subset of adult stem cells from bone marrow. The cells have multilineage differentiation potential and contribute to the regeneration of mesenchymal tissues such as bone, cartilage, fat, and muscle (Prockop, Science 276: 71-4 [1997]; Ferrari et al., Science 279: 1528-30 [1998]; Pittenger et al., Science 284: 143-7 [1999]; Dominici et al., Cytotherapy 8: 315-7 [2006]). Since MSCs are relatively easy to isolate and expand ex vivo, the cells have been used for tissue repair or regeneration in adult stem cell-based cell and gene therapy of a variety of diseases including osteogenesis imperfects, stroke, myocardiac infarction, pulmonary hypertension, and erectile dysfunction (Horwitz et al., Nat Med. 5: 309-13 [1999]; Zhang et al., Circ Res. 90: 284-8 [2002]; Toma et al., Circulation 105: 93-8 [2002]; Baber et al., Am J Physiol Heart Circ Physiol 292: H1120-8 [2007]; Deng et al., Am J Physiol Cell Physiol 285: C1322-29 [2003]; Deng et at., Stem Cells 22: 1279-91 [2004]; Deng et al., Int J Impot Res. 17 suppl 1: S57-63 [2005]; Deng et al., Life Sci. 78: 1830-8 [2006]; Bivalacqua et al., Am J Physiol Heart Circ Physiol. 292: H1278-90 [2007]). For example, MSCs have been shown to differentiate into osteoblasts, chondrocytes, myocytes, adipocytes, and beta-pancreatic islets cells. MSCs have a large capacity for self-renewal while maintaining their multipotency. MSCs can be isolated from bone marrow, umbilical cord blood, adipose tissue, and peripheral blood. Also, mesenchymal stem cells can be autologous, allogeneic, syngeneic, and xenogeneic mesenchymal stem cells, with respect to the individual or mammalian subject that is receiving the MSC treatment. The MSCs can be derived from human or other mammalian stem cells. The MSCs are genetically modified with extracellular superoxide dismutase using a vector to transfer the cDNA of extracellular superoxide dismutase into the MSCs for the production and secretion of extracellular superoxide dismutase by the MSCs.

A “protective amount of the ECSOD-MSCs” as used herein refers to that amount which is both non-toxic and creates the desired effect, wherein the desired effect is eliminating or reducing in severity or in extent the deleterious cellular effects caused by exposure to or treatment with ionizing radiation. The MSCs can be administered in a single or multiple dose administration to a subject or patient. A protective amount of the ECSOD-MSCs also refers to that amount which is effective, upon single or multiple dose administration to humans and other living organisms, in eliminating or reducing in severity or in extent the destructive cellular effects caused by exposure to ionizing radiation.

A protective amount of ECSOD-MSCs can be administered to a subject or a patient using techniques known to those of skill in the art and by observing results obtained under analogous circumstances. The protective amount of ECSOD-MSCs can be readily determined by one of ordinary skill in the art. In determining the protective amount or dose, a number of factors are considered by one skilled in the art, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances all of which are well known to those of skill in the art.

The term “vector” as used herein is intended to refer to a vehicle known in the art that can be manipulated by insertion or incorporation of a polynucleotide, for genetic manipulation (i.e., “cloning vectors”), or can be used to transcribe or translate the inserted polynucleotide (i.e., “expression vectors”). Such vectors are useful for introducing polynucleotides, including a nutrient-regulatable expression control element in operable linkage with a nucleic acid, and expressing the transcribed antisense or encoded protein in cells in vitro or in vivo. Examples of such vectors include, but are not limited to (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picarnovirus vectors; (i) vaccinia virus vectors; (j) a helper-dependent or gutless adenovirus; and (k) a plasmid.

The vector is therefore capable of transferring gene sequences to target cells (e.g., viral vectors, non-viral vectors, particulate carriers, and liposomes). Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors.

A transfer vector is a DNA molecule which contains, inter alia, genetic information that insures its own replication when transferred to a host microorganism strain. Examples of transfer vectors commonly used in bacterial genetics are plasmids and the DNA of certain bacteriophages.

“Plasmid” is the term applied to any autonomously replicating DNA unit which might be found in a microbial cell, other than the genome of the host cell itself. A plasmid is not genetically linked to the chromosome of the host cell. Plasmid DNA's exist as double stranded ring structures generally on the order of a few million daltons molecular weight, although some are greater than 10⁸ daltons in molecular weight. They usually represent only a small percent of the total DNA of the cell.

Transfer vector DNA is usually separable from host cell DNA by virtue of the great difference in size between them. Transfer vectors carry genetic information enabling them to replicate within the host cell, in some cases independently of the rate of host cell division. Some plasmids have the property that their replication rate can be controlled by the investigator by variations in the growth conditions.

Plasmid DNA exists as a closed ring. However, by appropriate techniques, the ring may be opened, a fragment of heterologous DNA inserted, and the ring reclosed, forming an enlarged molecule comprising the inserted DNA segment. Bacteriophage DNA may carry a segment of heterologous DNA inserted in place of certain nonessential phage genes. Either way, the transfer vector serves as a carrier or vector for an inserted fragment of heterologous DNA.

Transfer is accomplished by a process known as transformation. During transformation, bacterial cells mixed with plasmid DNA incorporate entire plasmid molecules into the cells. It is possible to maximize the proportion of bacterial cells capable of taking up plasmid DNA and hence of being transformed, by certain empirically determined treatments. Once a cell has incorporated a plasmid, the latter is replicated within the cell and the plasmid replicas are distributed to the daughter cells when the cell divides. Any genetic information contained in the nucleotide sequence of the plasmid DNA can, in principle, be expressed in the host cell.

Typically, a transformed host cell is recognized by its acquisition of traits carried on the plasmid, such as resistance to certain antibiotics. Different plasmids are recognizable by the different capabilities or combination of capabilities which they confer upon the host cell containing them. Any given plasmid may be made in quantity by growing a pure culture of cells containing the plasmid and isolating the plasmid DNA therefrom.

“Adenoviruses (Ad)” are double-stranded linear DNA viruses with a 36 kb genome. Several features of adenovirus have made them useful as transgene delivery vehicles for therapeutic applications, such as facilitating in vivo gene delivery.

“Lentiviral vector and recombinant lentiviral vector” refer to a nucleic acid construct which carries, and within certain embodiments, is capable of directing the expression of a nucleic acid molecule of interest. The lentiviral vector include at least one transcriptional promoter/enhancer or locus defining element(s), or other elements which control gene expression by other means such as alternate splicing, nuclear RNA export, post-translational modification of messenger, or post-transcriptional modification of protein. Such vector constructs can also include a packaging signal, long terminal repeats (LTRS) or portion thereof, and positive and negative strand primer binding sites appropriate to the retrovirus used (if these are not already present in the retroviral vector). The recombinant lentivirus is capable of reverse transcribing its genetic material (RNA) into DNA and incorporating this genetic material into a host cell's DNA upon infection. Lentiviral vector particles may have a lentiviral envelope, a non-lentiviral envelope (e.g., an ampho or VSV-G envelope), or a chimeric envelope.

A vector generally contains at least an origin of replication for propagation in a cell. Control elements, including expression control elements (e.g., nutrient-regulatable) as set forth herein, present within a vector, are included to facilitate transcription and translation. The term “control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, among others.

“Nucleic acid expression vector” or “Expression cassette” refers to an assembly which is capable of directing the expression of a sequence or gene of interest. The nucleic acid expression vector includes a promoter which is operably linked to the sequences or gene(s) of interest. Other control elements may be present as well. In addition to the components of the expression cassette, the plasmid construct may also include a bacterial origin of replication, one or more selectable markers, a signal which allows the plasmid construct to exist as single-stranded DNA (e.g., a M13 origin of replication), a multiple cloning site, and a “mammalian” origin of replication (e.g., a SV40 or adenovirus origin of replication).

Vectors can include a selection marker. As is known in the art, “selection marker” or equivalents means genes that allow the selection of cells containing the gene. “Positive selection” refers to a process whereby only the cells that contain the positive selection marker will survive upon exposure to the positive selection agent or be marked. For example, drug resistance is a common positive selection marker; cells containing the positive selection marker will survive in culture medium containing the selection drug, and those which do not contain the resistance gene will die.

“Expression control elements” include polynucleotides, such as promoters and enhancers that influence expression of an operably linked nucleic acid. Expression control elements and promoters include those active in a particular tissue or cell type, referred to herein as a “tissue-specific expression control elements/promoters.” Tissue-specific expression control elements are typically active in specific cells or tissues because they are recognized by transcriptional activator proteins, or other regulators of transcription, that are unique to a specific cell or tissue type.

Typical “control elements”, include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences. For example, the sequences and/or vectors described herein may also include one or more additional sequences that may optimize translation and/or termination including, but not limited to, a Kozak sequence (e.g., GCCACC placed in front (5′) of the ATG of the codon-optimized wild-type leader or any other suitable leader sequence (e.g., tpa1, tpa2, wtLnat (native wild-type leader)) or a termination sequence (e.g., TAA or, preferably, TAAA placed after (3′) the coding sequence

The current embodiment provides a method for treating radiation damage in a mammalian subject or a human individual is disclosed which entails in vivo administration of MSCs genetically modified with ECSOD to the subject. The genetically modified mesenchymal stem cells are capable of secreting extracellular superoxide dismutase to neutralize or eliminate the toxic superoxide anion that is elicited by ionizing radiation. The ECSOD-MSCs are administered in a therapeutically acceptable amount to a subject in need of treatment. The method is particularly useful for the treatment of radiation damage after radiation exposure as a consequence of radiation accident, nuclear accident, nuclear terrorist attack, nuclear war, other radiological emergencies, space travel, radiation therapy, and diagnostic radiology. It can also be administered prior to planned radiation exposure in order to minimize its potential toxic effects or allow higher therapeutic doses to targeted diseased areas to be administered without side effects to surrounding normal tissues.

The method is also useful for the prevention and prophylactic treatment of radiation damage before radiation exposure as a consequence of radiation accident, nuclear accident, nuclear terrorist attack, nuclear war, other radiological emergencies, space travel, radiation therapy, and diagnostic radiology. For example, subjects that are likely to enter into a location susceptible to such attacks can receive a preventative treatment. This will either prevent or at least may limit the damage caused by the exposure to radiation. In another embodiment, the treatment is provided to pregnant women either before or after an X-ray to prevent or treat damage resulting from the radiation exposure.

The method is further useful for the treatment of radiation damage to normal tissues after radiation therapy in tumor patients. The ECSOD-MSCs treatment can be used to treat radiation damage to normal tissues after radiation therapy in tumor patients. The ECSOD-MSCs treatment can decrease the severity of damage to normal tissues, particularly bone marrow, spleen, gastrointestinal tract, and healthy tissues near the tumor, caused by radiation therapy. The method is also useful for the prevention and prophylactic treatment of radiation damage to normal tissues before radiation therapy in tumor patients.

The current embodiment provides a method that protects cells and living organisms from deleterious cellular effects related either to exposure to radiation or exposure to other substances that cause damage similar to radiation. The treatment functions by preventing or eliminating the harmful effects or by reducing their severity. The subject to be protected can be administered the ECSOD-MSCs of the current embodiment prior to, during, or after exposure of the cell to radiation or other substances that cause damage in a similar fashion like chemotherapy. The ECSOD-MSCs of the current embodiment can provide a protective effect in the cell and the subject by eliminating or reducing the severity of the detrimental cellular effects that would otherwise be caused by the exposure. Therefore, the ECSOD-MSCs of the current embodiment enable survival or lengthen survival of living organisms in otherwise lethal conditions. ECSOD-MSCs may also decrease morbidities under otherwise sublethal conditions.

More particularly, the current embodiment provides a method of protecting non-cancer, or normal, cells of a subject from deleterious cellular effects caused by exposure of the mammal to ionizing radiation. The ECSOD-MSCs of the current embodiment provide a protection of normal cells during exposure to radiation, such as during radiation therapy or diagnostic procedures such as x-rays and CAT scans. The cancer cells, if protected at all, are protected to a lesser extent than normal cells. The current embodiment provides a method whereby the deleterious cellular effects on non-cancer cells caused by exposure of the mammal to radiation are eliminated or reduced in severity or in extent. This treatment enables greater amounts of radiation to be administered to a patient without the detrimental side effects. In another embodiment, ECSOD-MSCs can protect the eyes against the cataract that develops as a result of the toxic effects of radiation on the lens.

Additionally, the ECSOD-MSCs can be administered in a higher dose to provide a systemic protective effect. The benefit of the systemic effect is that a dose of ECSOD-MSCs can be administered to a patient and provide the desired effect at a variety of locations. This alleviates the need to locate all locations in need of treatment. The ECSOD-MSCs can be administered via intravenous administration, intra-bone marrow administration, intra-arterial administration, intra-cardiac injection, intracerebral injection, intraspinal injection, intra-peritoneal injection, intra-muscular injection, subcutaneous injection, parenteral administration, intra-rectal administration, intra-tracheal injection, intra-nasal administration, intradermal injection, and the like. The ECSOD-MSCs can be administrated to the human individual or mammalian subject systemically, at the site of injury, at an adjacent site to the site of injury, and where following administrating the cells migrate to the site of injury.

The ECSOD-MSCs can be administered to the human or other animal after irradiation in an amount that is effective for diminishing damage to the respiratory, gastrointestinal, hematopoietic, or other systems after sublethal irradiation or for increasing the survival rate after lethal irradiation. The ECSOD-MSCs are also effective when administered prior to or during exposure to radiation.

The ECSOD-MSCs may be administered as single doses or as multiple doses and are ordinarily administered prior to, during, or after exposure to radiation. The ECSOD-MSCs will be administered in single or multiple doses prior to, during, or after radiation therapy following a schedule calculated to provide the maximum selective protective effect during radiation therapy or substances that cause similar damage such as chemotherapy as can be determined by those of skill in the art. The ECSOD-MSCs can also be administered in conjunction with other therapeutic agents.

The details of the dosing schedule for the ECSOD-MSCs that provide the maximum selective protective effect upon exposure to ionizing radiation can be readily determined by one skilled in the art by the use of known techniques and by observing results obtained under analogous circumstances.

A protective amount of the ECSOD-MSCs for administration to a mammal or patient will vary depending upon the amount of radiation exposure and the time period of radiation exposure, with the upper limit of the composition limited by the toxicity of a large dose. A larger dose of the ECSOD-MSCs will be required for lethal radiation exposure, while a lower dose can be used where the radiation exposure is sub-lethal or chronic.

The ECSOD-MSCs of the current embodiment can be administered to a mammal, a healthy individual, or a patient in any form or mode that makes the ECSOD-MSCs available in effective amounts. For example, the composition of the current embodiment can be administered intravenously, subcutaneously, intramuscularly, intraperitoneally, transdermally, intranasally, rectally, and the like. One skilled in the art of preparing formulations can readily select the proper form and mode of administration depending upon the particular characteristics of the compound selected the disease state to be treated, the stage of the disease, and other relevant circumstances.

The ECSOD-MSCs are formed using standard transplantation and transfection protocols, examples of which are detailed below. The MSCs are genetically altered to secrete ECSOD by introducing DNA into the stem cells using a gene that encodes for ECSOD. For example, the mesenchymal stem cells are genetically modified with a vector. The vector can be introduced into the mesenchymal stem cells, for example, by transduction. Transduction is the introduction of foreign DNA into a cell using a viral vector. Suitable methods are well know to one skilled in the art and some suitable methods are described above/below. Preferably, the vector comprises a gene which encodes for superoxide dismutase.

In one embodiment, transplantation includes the steps of isolating a stem cell and transferring the ECSOD-MSCs into the patient. Transplantation can include transferring the ECSOD-MSCs into the patient by injection of a cell suspension into the patient, surgical implantation of a mass of the ECSOD-MSCs into a tissue or organ of the patient, or perfusion of a tissue or organ with a cell suspension. The route of transferring or transplanting the ECSOD-MSCs is determined by the need for the cell to reside in a particular tissue or organ and by the ability of the cell to find and be retained by the desired target tissue or organ. In the case where a transplanted cell is to reside in a particular location, it can be surgically placed into a tissue or organ or simply injected into the bloodstream if the cell has the capability to migrate to the desired target organ.

In another embodiment, the transplantation includes the steps of isolating the MSCs, culturing the MSCs, transferring ECSOD into MSCs, and transferring the ECSOD-MSCs into a mammal or a patient. The culturing step can include a variety of MSC culturing procedures as are well known to those of skill in the art.

In another embodiment, the transplantation can include the steps of isolating the ECSOD-MSCs as described herein, differentiating the ECSOD-MSCs, and transferring the ECSOD-MSCs into a mammal or a patient. The differentiating step will vary depending upon the MSCs used as well as the intended use. Examples of such differentiating protocols are well known to those of skill in the art. The transplantation, can further include the expanding the ECSOD-MSCs during the differentiating step. Expansion protocols are well known to those of skill in the art.

A variety of methods are available for gene transfer into stem cells. Calcium phosphate precipitated DNA has been used, but provides a low efficiency of transformation, especially for nonadherent cells. In addition, calcium phosphate precipitated DNA methods often result in insertion of multiple tandem repeats, increasing the likelihood of disrupting gene function of either endogenous or exogenous DNA (Boggs, 1990). The use of cationic lipids, e.g., in the form of liposomes, is also an effective method of packaging DNA for transfecting eukaryotic cells, and several commercial preparations of cationic lipids are available. Electroporation provides improved transformation efficiency over the calcium phosphate protocol. It has the advantage of providing a single copy insert at a single site in the genome. Direct microinjection of DNA into the nucleus of cells is yet another method of gene transfer. It has been shown to provide efficiencies of nearly 100% for short-term transfection, and 20% for stable DNA integration. Microinjection bypasses the sometimes-problematic cellular transport of exogenous DNA through the cytoplasm. The protocol requires small volumes of materials. It allows for the introduction of known amounts of DNA per cell. The ability to obtain a virtually pure population of MSCs would improve the feasibility of the microinjection approach to targeted gene modification of mesenchymal stem cells. Microinjection is a tedious, highly specialized protocol. The very nature of the protocol limits the number of cells that can be injected at any given time, making its use in large-scale production limited. Gene insertion into MSCs using retroviral methods is the preferred method. Retroviruses provide a random, single-copy, single-site insert at very high transfection efficiencies. Other such transfection methods are known to one skilled in the art and are considered to be within the scope of this invention.

In another embodiment, the gene transfer protocols involve retroviral vectors as the “helper virus” (i.e., encapsulation-defective viral genomes that carry the foreign gene of interest but are unable to form complete viral particles). Other carriers such as DNA-mediated transfer, adenovirus, SV40, adeno-associated virus, and herpes simplex virus vectors can also be employed. Several factors can be considered when selecting the appropriate vector for infection. It is sometimes preferable to use a viral long terminal repeat or a strong internal promoter to express the foreign gene rather than rely on spliced subgenomic RNA.

The two primary methods of MSC transduction are co-culture and supernatant infection. Supernatant infection involves repeated exposure of MSCs to the viral supernatant. Co-culture involves the comingling of MSCs and an infected “package cell line” for periods of 24 to 48 hours. Co-culture is typically more efficient than supernatant infection for MSC transduction. After co-culture, infected MSCs are often further cultured to establish a long term culture (LTC).

The cell line containing the helper virus is referred to as the package cell line. A variety of package cell lines are currently available. One feature of the package cell line is that it does not produce replication-competent helper virus.

In one embodiment of the invention animals or patients from whom MSCs are harvested may be treated with 5-fluorouracil (5-FU) prior to extraction. 5-FU treated MSCs are more susceptible to retroviral infection than untreated cells. 5-FU MSCs dramatically reduce the number of clonogenic progenitors, however.

In another embodiment, harvested MSCs may be exposed to various growth factors, such as those employed to promote proliferation or differentiation of mesenchymal stem cells. Growth factors can be introduced in culture before, during, or after infection to improve cell replication and transduction. Studies report the use of growth factors increase transformation efficiency from 30 to 80%.

In a further embodiment, a replicable expression vector is used that includes a DNA sequence encoding ECSOD. In the present context, the term “replicable” means that the vector is able to replicate in a given type of host cell into which it has been introduced. The vector may be one carrying the DNA sequence shown above or any suitable modification thereof as explained above. Immediately upstream of this sequence (the coding sequence of ECSOD) there may be provided a sequence coding for a signal peptide, the presence of which ensures secretion of the ECSOD expressed by host cells harboring the vector. It should be noted that this signal sequence (and the signal peptide encoded by it) in itself forms an aspect of the current embodiment, and it is contemplated that it may be inserted upstream of DNA sequences coding for other proteins or peptides so as to obtain secretion of the resulting products from the ECSOD-MSCs.

The vector may be any vector that may conveniently be subjected to recombinant DNA procedures, and the choice of vector will often depend on the host cell into which it is to be introduced. Thus, the vector may be an autonomously replicating vector, i.e. a vector that exists as an extrachromosomal entity, the replication of which is independent of chromosomal replication; examples of such a vector are a plasmid, phage, cosmid, mini-chromosome or virus. Alternatively, the vector may be one which, when introduced in a host cell, is integrated in the host cell genome and replicated together with the chromosome(s) into which it has been integrated.

In a further aspect, the invention relates to a cell line that is capable of secreting ECSOD. While various human cell lines derived from a wide variety of tissue cells as well as tumor cell lines have previously been analyzed for their content of ECSOD (cf. Marklund, J. Clin. Invest. 74, October 1984, pp. 1398-1403), no conclusive results were obtained.

The current embodiment further relates to an MSC harboring a replicable expression vector as defined above. In principle, the cell may be of any type of cell, i.e. a prokaryotic cell such as a bacterium, a unicellular eukaryotic organism, a fungus or yeast, or a cell derived from a multicellular organism, e.g. an animal or a plant. It is, however, believed that a mammalian cell may be particularly capable of expressing ECSOD, which is, after all, a highly complex molecule, which cells of lower organisms, might not be able to produce.

In another embodiment, a nucleic acid expression construct used in the invention is designed to target production of proteins in gastrointestinal endocrine cells. The construct contains an expression control element operably linked to desired nucleic acid sequences. Expression control elements include promoters capable of targeting expression of a linked nucleic acid of interest to endocrine cells in the gut. Introduction of constructs into target cells can be carried out by conventional methods well known in the art (osmotic shock (e.g., calcium phosphate), electroporation, viral vectors, vesicles or lipid carriers (e.g., lipofection), direct microinjection, etc.).

As shown above, administration of mesenchymal stem cells genetically modified with extracellular superoxide dismutase (ECSOD-MSCs) improves survival in irradiated mice (Abdel-Mageed et al., Blood 113: 1201-3 [2009]). Since ECSOD-MSCs can be used to treat acute radiation syndrome, in particular the radiation-induced gastrointestinal damage, irradiated mice treated with ECSOD-MSCs can be used as a model organism for intestinal stem cell study.

The existence of other types of stem cells has been known for decades. For example, there are numerous “organ specific stem cells,” such as intestinal epithelial stem cells, hematopoietic stem cells, pancreatic stem cells, cardiac stem cells, prostate stem cells, kidney stem cells, eye stem cells, lung stem cells, liver stem cells, and neural stem cells are known to exist, but there remains a lack of a good model organism. The existence of intestinal epithelial stem cells has been known for decades, but the cells have not been isolated for characterization and experimentation due to lack of a good model organism. Also, the relationship between radiation damage and intestinal epithelial stem cells is well known. A murine radiation-induced intestinal injury model is frequently used for the study of intestinal stem cells, which suffers from the impact of oxidative stress after radiation exposure, and does not allow sufficient time for recovery of radiation-injured intestinal stem cells. Irradiated tissues release superoxide anion (O₂⁻) for months after radiation exposure, which is a major cause for radiation-induced cell apoptosis. Radiation exposure induces oxidative damage to bone marrow and gastrointestinal tract and causes bone marrow failure and gastrointestinal syndrome.

ECSOD-MSCs can enhance recovery of irradiated mice due to a reduction in the injury from O₂⁻ in the irradiated gastrointestinal tract and/or bone marrow. This creates a superior ability to isolate, culture, and characterize intestinal stem cells. Therefore, the intravenous administration of ECSOD-MSCs after total body radiation exposure can enhance the recovery of radiation-injured intestinal stem cells. Additionally, the intravenous administration of ECSOD-MSCs after abdominal radiation exposure can enhance the recovery of radiation-injured intestinal stem cells. Finally, the information enables the ability to isolate, culture, characterize, functionally validate, and compare stem cell populations from the small intestinal epithelium in vivo and in vitro using irradiated mice treated with ECSOD-MSCs.

The mouse model can accelerate research on organ specific stem cells. Improved therapies for the related diseases, such as radiation injury, inflammation, and cancer may be developed based on better understanding of the organ specific stem cells.

Formation of superoxide anion (O₂⁻) after ionizing radiation is a major determinant of the lethality of whole-body radiation exposure. Irradiated tissues release O₂⁻ for anywhere from hours to months after radiation exposure, which is a major cause for radiation-induced cell apoptosis. Radiation exposure induces oxidative damage to the bone marrow and the gastrointestinal tract and causes bone marrow failure and gastrointestinal syndrome. Extracellular superoxide dismutase (ECSOD) is a potent antioxidant enzyme catalyzing the dismutation of ECSOD and has been used in gene therapy of diseases involving oxidative stress.

This model of ECSOD-MSCs enhanced recovery of irradiated mice due to a reduction in the injury from O₂⁻ in the irradiated gastrointestinal tract and/or bone marrow provides for superior ability to isolate, culture, and characterize intestinal stem cells in a murine model. Therefore, the ECSOD-MSCs for radioprotection approach described herein provide a feasible model for intestinal stem cell study in mice.

The current embodiment enables stem cell populations from the small intestinal epithelium to be isolated, cultured, characterized, functionally validated, and compared in vivo and in vitro.

The results of the studies included in the Examples increase the understanding and knowledge of the effect of ECSOD-MSCs on intestinal stem cell recovery after radiation exposure and accelerate the research on stem cells of the small intestine, such as identification, isolation, and characterization. These results create a model organism for intestinal stem cell study, which can greatly facilitate understanding of the biology and function of the intestine and aid development of therapies for intestinal diseases and conditions where damage and replacement of intestinal epithelium are components, including radiation injury, inflammation, and cancer.

The above discussion provides a factual basis for the use of the ECSOD-MSCs of the current embodiment. The method used with and the utility of the current embodiment can be further shown by the following non-limiting examples and accompanying figures.

### EXAMPLES

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds.) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

### Example 1

### Materials and Methods

### Adenoviral Vectors

The following two adenoviral vectors were used:

(1). Ad5CMVECSOD: a replication-deficient recombinant adenovirus carrying the human extracellular superoxide dismutase (ECSOD) gene under the control of cytomegalovirus (CMV) promoter (Chu et al. Circ Res. 92:461-8 [2003]).
(2). Ad5CMVntlacZ: a replication-deficient recombinant adenovirus carrying the nuclear-targeted β-galactosidase reporter gene ntlacZ under the control of CMV promoter (Chu et al. Circ Res. 92:461-8 [2003]).

Both adenoviral vectors were purchased from University of Iowa Gene Transfer Vector Core (Iowa City, Iowa).

Isolation and Ex Vivo Expansion of Mouse Mesenchymal Stem Cells (mMSCs)

mMSCs were isolated as previously described (Deng et al., Am J Physiol Cell Physiol 285:C1322-9 [2003]; Sun et al., Stem Cells. 21:527-35 [2003]; Peister et al. Blood 103:1662-8 [2004]; Bivalacqua et al. Am J Physiol Heart Circ Physiol. 292:H1278-90 [2007]; Abdel-Mageed et al., Blood 113: 1201-3 [2009]). Six-week-old female BALB/c mice (The Jackson Laboratory, Bar Harbor, Me.), were euthanized with CO₂ and femurs and tibias were removed. Both ends of the bones were cut and bone marrow was flushed out using a 18-gauge needle and culture medium for mMSCs [MEM-α (Atlanta Biologicals, Norcross, Ga.); 20% fetal bovine serum (FBS, GIBCO Invitrogen Corp., Carlsbad, Calif.); 100 units/ml penicillin, 100 μg/ml streptomycin, and 250 ng/ml amphotericin B (Atlanta Biologicals); and 2 mM L-glutamine (GIBCO Invitrogen Corp)]. The bone marrow cells were filtered through a cell strainer with 70-μm nylon mesh (BD Bioscience, Bedford, Mass.), and the cells from each mouse were plated in a T75 flask (Falcon, Fisher Scientific, Pittsburgh, Pa.). The cells were incubated at 37° C. with 5% humidified CO₂, and mMSCs were isolated by their adherence to tissue culture plastic. Fresh culture medium was added and replaced every 2-3 days. The adherent mMSCs were grown to 90% confluence, harvested with 0.25% trypsin/1 mM EDTA for 2 minutes at 37° C., and diluted 1:3 for ex vivo expansion.

Adenoviral Transduction of mMSCs to Secrete Biologically Active ECSOD

mMSCs were transduced with adenoviral vectors as previously described (Deng et al., Stem Cells 22: 1279-91 [2004]; Baber et al., Am J Physiol Heart Circ Physiol. 292: H1120-8 [2007]; Abdel-Mageed et al., Blood 113: 1201-3 [2009])). Briefly, mMSCs were plated at a density of 10,000 cells/cm² in 6-well plates or T75 flasks (Falcon, Fisher Scientific) and incubated overnight. The cells were counted and then exposed to fresh culture medium containing Ad5CMVECSOD at 0, 300, or 2000 multiplicities of infection (MOI) for 48 hours. MOI is defined as pfu/cell. Virus-containing culture medium was discarded, cells were washed 3 times with PBS, and fresh culture medium was added. Cells were counted, cultured for 48 hours, and culture supernatant was collected. The culture supernatant was then assayed for the secretion of biologically active ECSOD by Ad5CMVECSOD-transduced mMSCs using a SOD activity assay kit (Cayman Chemical Company, Ann Arbor, Mich.).

Adenoviral Transduction of mMSCs to Express β-Galactosidase

mMSCs were transduced with adenoviral vectors as previously described (Deng et al., Am J Physiol Cell Physiol. 285:C1322-9 [2003]; Deng et al., Life Sci. 78: 1830-8 [2006]; Abdel-Mageed et al., Blood 113: 1201-3 [2009]). Briefly, mMSCs were plated at a density of 10,000 cells/cm² in 6-well plates or T75 flasks (Falcon, Fisher Scientific) and incubated overnight. The cells were counted and then exposed to fresh culture medium containing Ad5CMVntlacZ at 0, 300, or 2000 MOI for 48 hours. To conduct X-gal cytochemistry for β-galactosidase activity, the transduced mMSCs were washed with PBS, fixed for 5 minutes in fixing solution (2% formaldehyde, 0.2% glutaraldehyde, Sigma, St. Louis, Mo.), washed twice with PBS, and incubated in staining solution (1 mg/ml X-gal, 5 mM K ferricyanide, 5 mM K ferrocyanide, and 2 mM MgCl₂, Sigma) at 37° C. in the dark overnight. Cells were washed with PBS and the expression of transgene ntlacZ in mMSCs was evaluated by light microscopy scoring of blue cells expressing the nuclear-targeted β-galactosidase activity (Deng et al. Stem Cells 22:1279-91 [2004]; Abdel-Mageed et al., Blood 113: 1201-3 [2009]).

In Vitro Differentiation of mMSCs into Osteoblasts or Adipocytes

In vitro differentiation of mMSCs into osteoblast or adipocyte lineages was conducted as previously described (Deng et al., Am J Physiol Cell Physiol 285:C1322-9 [2003]; Deng et al. Stem Cells. 22:1279-91 [2004]). Cells in 6-well plates or T75 flasks were treated with culture medium for mMSCs plus either osteogenic supplement (1×10⁻⁵ mM dexamethasone, 0.2 mM ascorbic acid, and 10 mM β-glycerol phosphate, Sigma) or adipogenic supplement (0.5 μM hydrocortisone, 500 μM isobutylmethylxanthine, and 60 μM indomethacin, Sigma). The differentiation medium was changed every 3 days until day 21. To assess mineral deposition, cells were washed with PBS, fixed with cold methanol (−20° C.) for 10 minutes, washed with dH₂O twice, stained with 2% Alizarin red S (pH 4.1, Sigma) for 15 minutes, washed with dH₂O five times, and checked under an inverted phase contrast microscope. To assess lipid droplet formation, cells were washed with PBS, fixed with 10% formalin (Sigma) for 1 hour, washed with dH₂O twice, stained with a freshly prepared Oil red 0 solution for 15 minutes, washed with dH₂O, and checked under an inverted phase contrast microscope. The Oil red O solution was prepared by mixing three parts of an Oil red O stock solution (0.5%, prepared in isopropanol, Sigma) with two parts of dH₂O and filtering through a 0.45 μm pore size filter.

Removal of Cell Clumps in Mouse Mesenchymal Stem Cell (mMSC) Suspension by Filtration Method

mMSCs were suspended in phosphate buffered saline (PBS) at a concentration of 2.5×10⁶ cells/ml. The cells were then filtered through a cell strainer with 40 μm nylon mesh (BD Biosciences, Bedford, Mass.) to remove cell clumps. A phase contrast microscope was used for the observation of cells before and after filtration.

Isolation and Culture of Human Mesenchymal Stem Cells (hMSCs)

hMSCs were isolated as previously described (Deng et al. Biochem Biophys Res Commun 282:148-52 [2001]; Mageed et al. Transplantation 83:1019-26 [2007]). About 10 ml bone marrow aspirate were taken from the iliac crest of normal donors. The bone marrow aspirate was diluted 1:1 with Hanks' Balanced Salt Solution (HBSS, GIBCO Invitrogen Corp.) and layered over 15 ml Ficoll (Fico/Lite LymphoH, Atlanta Biologicals). After centrifugation at 400×g for 30 minutes, the mononuclear cell layer was recovered from the gradient interface and washed with HBSS. The cells were suspended in 10 ml culture medium for hMSCs [MEM-α (Atlanta Biologicals); 20% fetal bovine serum (FBS, GIBCO Invitrogen Corp.); 100 units/ml penicillin and 100 μg/ml streptomycin (Atlanta Biologicals); and 2 mM L-glutamine (GIBCO Invitrogen Corp.)]. All of the cells were plated in one T75 flask (Falcon, Fisher Scientific) and incubated at 37° C. with 5% humidified CO₂. Three days later, the culture medium was discarded to remove non-adherent cells and hMSCs were isolated by their adherence to tissue culture plastic. Fresh culture medium was added and replaced every 2-3 days. The adherent hMSCs were grown to 70-90% confluency over about 14 days. The cells were harvested with 0.25% Trypsin/1 mM EDTA for 5 minutes at 37° C. and diluted 1:3 for ex vivo expansion.

Adenoviral Transduction of hMSCs to Secrete Biologically Active ECSOD

hMSCs were transduced with adenoviral vectors as previously described (Deng et al., Stem Cells 22: 1279-91 [2004]; Baber et al., Am J Physiol Heart Circ Physiol. 292: H1120-8 [2007]). hMSCs were plated at a density of 10,000 cells/cm² in 6-well plates or T75 flasks and incubated overnight. The cells were counted and then exposed to fresh culture medium containing Ad5CMVECSOD at 0, 300, or 2000 MOI for 48 hours. Virus-containing culture medium was discarded, cells were washed 3 times with PBS, and fresh culture medium was added. Cells were then counted, cultured for 48 hours, and culture supernatant was collected. The culture supernatant was then assayed for the secretion of biologically active ECSOD by Ad5CMVECSOD-transduced hMSCs using a SOD activity assay kit (Cayman Chemical Company).

Adenoviral Transduction of hMSCs to Express β-Galactosidase

hMSCs were transduced with adenoviral vectors as previously described (Deng et al., Am J Physiol Cell Physiol 285:C1322-9 [2003]. Deng et al., Life Sci. 78: 1830-8 [2006]). hMSCs were plated at a density of 10,000 cells/cm² in 6-well plates or T75 flasks (Falcon, Fisher Scientific) and incubated overnight. The cells were counted and then exposed to fresh culture medium containing Ad5CMVntlacZ at 0, 300, or 2000 MOI for 48 hours.

To conduct X-gal cytochemistry for β-galactosidase activity, the transduced hMSCs were washed with PBS, fixed for 5 minutes in fixing solution (2% formaldehyde, 0.2% glutaraldehyde, Sigma), washed twice with PBS, and incubated in staining solution (1 mg/ml X-gal, 5 mM K ferricyanide, 5 mM K ferrocyanide, and 2 mM MgCl₂, Sigma) at 37° C. in the dark overnight. Cells were washed with PBS and the expression of transgene ntlacZ in hMSCs was evaluated by light microscopy scoring of blue cells expressing the nuclear-targeted β-galactosidase activity (Deng et al. Stem Cells 22:1279-91 [2004]).

In Vitro Differentiation of hMSCs into Osteoblasts or Adipocytes

In vitro differentiation of hMSCs into osteoblast or adipocyte lineages was conducted as previously described (Deng et al. Biochem Biophys Res Commun. 282:148-52 [2001]; Deng et al., Am J Physiol Cell Physiol. 285:C1322-9 [2003]. Deng et al., Stem Cells. 22:1279-91 [2004]). Cells in 6-well plates or T75 flasks (Falcon, Fisher Scientific) were treated with culture medium for hMSCs plus either osteogenic supplement (1×10⁻⁵ mM dexamethasone, 0.2 mM ascorbic acid, and 10 mM β-glycerol phosphate, Sigma) or adipogenic supplement (0.5 μM hydrocortisone, 500 μM isobutylmethylxanthine, and 60 μM indomethacin, Sigma). The differentiation medium was changed every 3 days until day 21.

To assess mineral deposition, cells were washed with PBS, fixed with cold methanol (−20° C.) for 10 minutes, washed with dH₂O twice, stained with 2% Alizarin red S (pH 4.1, Sigma) for 15 minutes, washed with dH₂O five times, and checked under an inverted phase contrast microscope.

To assess lipid droplet formation, cells were washed with PBS, fixed with 10% formalin (Sigma) for 1 hour, washed with dH₂O twice, stained with a freshly prepared Oil red O solution for 15 minutes, washed with dH₂O, and checked under an inverted phase contrast microscope. The Oil red O solution was prepared by mixing three parts of an Oil red O stock solution (0.5%, prepared in isopropanol, Sigma) with two parts of dH₂O and filtering through a 0.45 μm pore size filter.

Intravenous Administration of ECSOD or ntlacZ Gene-Modified mMSCs into Irradiated Mice Through Tail Vein Injection

Five-week-old female BALB/c mice were given 9 Gy total body γ irradiation from a ¹³⁷Cs source (Gammacell 1000; MDS Nordion, Ottawa, ON) at a dose rate of 1.23 Gy/min. Twenty-four hours later, these animals received a tail vein injection of 200 μl phosphate buffered saline (PBS), 0.5×10⁶ ntlacZ gene-modified mMSCs in 200 μl PBS, or 0.5×10⁶ ECSOD gene-modified mMSCs in 200 μl PBS. All in vivo experiments were performed on mice in accordance with institutional and NIH guidelines for the care and use of laboratory animals.

To prepare ECSOD or ntlacZ gene-modified mMSCs, i.e. Ad5CMVECSOD or Ad5CMVntlacZ-transduced mMSCs, mMSCs were transduced with Ad5CMVECSOD or Ad5CMVntlacZ at MOI 2000 for 48 hours. The virus-containing culture medium was removed and the cells were washed 3 times with PBS. The Ad5CMVECSOD or Ad5CMVntlacZ-transduced mMSCs were then harvested with 0.25% Trypsin/1 mM EDTA, washed with PBS, and a cell suspension at a concentration of 2.5×10⁶ cells/ml was prepared in PBS for tail vein injection. For intravenous administration of PBS, Ad5CMVECSOD or Ad5CMVntlacZ-transduced mMSCs into the irradiated mice, 200 μl of PBS or 200 μl of cell suspension were injected into the tail vein. A total of 0.5×10⁶ cells or 200 μl PBS was injected into each mouse. The mice were then monitored 35 days for survival or death.

### Statistical Analysis

Data were expressed as mean±SEM (n=3) and were analyzed statistically using a one-way analysis of variance (ANOVA) followed by post hoc analysis with Tukey test. Kaplan-Meier survival curve was used for mouse survival data analysis.

### Results

Secretion of Biologically Active ECSOD by mMSCs Genetically Modified with ECSOD

To ascertain whether Ad5CMVECSOD can infect mMSCs and whether mMSCs genetically modified with ECSOD, also known as ECSOD gene-modified mMSCs or Ad5CMVECSOD-transduced mMSCs, can secrete functional ECSOD, mMSCs were transduced with Ad5CMVECSOD at MOI 0, 300 or 2000 for 48 hours. The cells were washed with PBS and further incubated for 48 hours. The culture supernatant was then collected and analyzed for SOD activity.

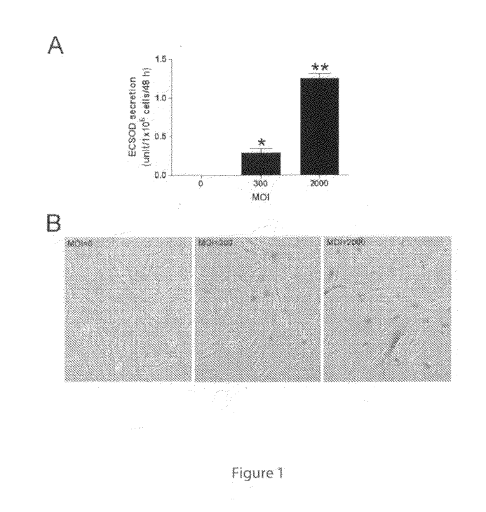

FIG. 1A demonstrates a dose-dependent secretion of biologically active ECSOD by Ad5CMVECSOD-transduced mMSCs. The efficacy of adenoviral-mediated gene transfer into mMSCs was further examined using the reporting gene ntlacZ. mMSCs were transduced with Ad5CMVntlacZ at MOI 0, 300, or 2000. After 48 hours, the expression of nuclear-targeted β-galactosidase in Ad5CMVntlacZ-transduced mMSCs was assessed by X-gal staining. As shown in FIG. 1B, transduction efficiency of Ad5CMVntlacZ into mMSCs is proved to be dose-dependent. Therefore, adenoviral transduction of mMSCs is effective and ECSOD gene-modified mMSCs produce and secrete biologically active ECSOD.

More specifically, FIG. 1A is a graph depicting secretion of biologically active extracellular superoxide dismutase (ECSOD) by Ad5CMVECSOD-transduced mMSCs. mMSCs were transduced with Ad5CMVECSOD at MOI 0, 300 or 2000 for 48 hours, virus-containing culture medium was removed and cells were washed 3 times with PBS and further incubated in fresh culture medium for 48 hours. The culture supernatant was collected and analyzed for superoxide dismutase (SOD) activity using a Cayman SOD activity assay kit. Data were expressed as mean±SEM (n=3) and analyzed statistically using a one-way analysis of variance (ANOVA) followed by post hoc analysis with Tukey test. * P<0.05 versus MOI 0, ** P<0.05 versus MOI 0 or 300. FIG. 1B is a set of images depicting expression of nuclear targeted β-galactosidase by Ad5CMVntlacZ-transduced mMSCs. mMSCs were transduced with Ad5CMVntlacZ at MOI 0, 300, or 2000 for 48 hours. The cells were then X-gal stained for β-galactosidase activity and the blue nuclear-targeted β-galactosidase positive Ad5CMVntlacZ-transduced mMSCs were identified. Original magnification: ×40.

Secretion of Biologically Active ECSOD by hMSCs Genetically Modified with ECSOD

To test the hypothesis that MSCs genetically modified with ECSOD have a radioprotective effect, human MSCs (hMSCs) were isolated by their adherence to tissue-culture plastic from healthy bone marrow donors and ex vivo expanded as previously described (Deng et al. Biochem Biophys Res Commun 282:148-52 [2001]; Mageed et al. Transplantation 83:1019-26 [2007]).

To ascertain whether Ad5CMVECSOD can infect hMSCs and whether hMSCs genetically modified with ECSOD, also known as ECSOD gene-modified hMSCs or Ad5CMVECSOD-transduced hMSCs, can secrete functional ECSOD, hMSCs were transduced with Ad5CMVECSOD at MOI 0, 300 or 2000 for 48 hours. The cells were washed with PBS and further incubated for 48 hours. The culture supernatant was then collected and analyzed for SOD activity.

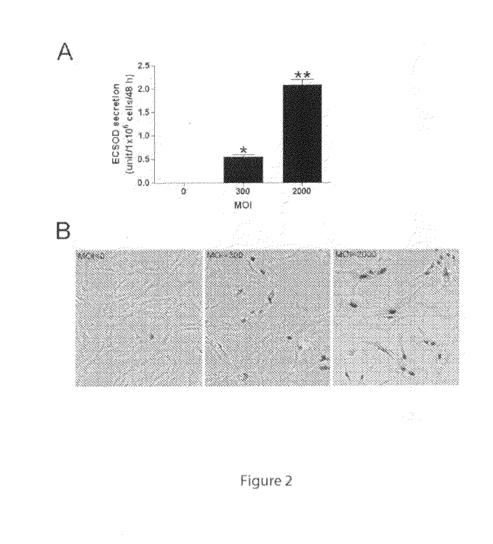

FIG. 2A demonstrates a dose-dependent secretion of biologically active ECSOD by Ad5CMVECSOD-transduced hMSCs. The efficacy of adenoviral-mediated gene transfer into hMSCs was further examined using the reporting gene ntlacZ. To this end, hMSCs were transduced with Ad5CMVntlacZ at MOI 0, 300, or 2000. After 48 hours, the expression of nuclear-targeted β-galactosidase in Ad5CMVntlacZ-transduced hMSCs was assessed by X-gal staining. As shown in FIG. 2B, transduction efficiency of Ad5CMVntlacZ into hMSCs is proved to be dose-dependent. Therefore, adenoviral transduction of hMSCs is effective and ECSOD gene-modified hMSCs produce and secrete biologically active ECSOD.

More specifically, FIG. 2A is a graph depicting secretion of biologically active ECSOD by Ad5CMVECSOD-transduced hMSCs. hMSCs were transduced with Ad5CMVECSOD at MOI 0, 300 or 2000 for 48 hours, virus-containing culture medium was removed and cells were washed 3 times with PBS and further incubated in fresh culture medium for 48 hours. The culture supernatant was collected and analyzed for SOD activity using a Cayman SOD activity assay kit. Data were expressed as mean±SEM (n=3) and analyzed statistically using a one-way ANOVA followed by post hoc analysis with Tukey test. * P<0.01 versus MOI 0, ** P<0.001 versus MOI 0 or 300. FIG. 2B is a set of images depicting expression of nuclear targeted β-galactosidase by Ad5CMVntlacZ-transduced hMSCs. hMSCs were transduced with Ad5CMVntlacZ at MOI 0, 300, or 2000 for 48 hours. The cells were then X-gal stained for β-galactosidase activity and the blue nuclear-targeted β-galactosidase positive Ad5CMVntlacZ-transduced hMSCs were identified. Original magnification, ×40.

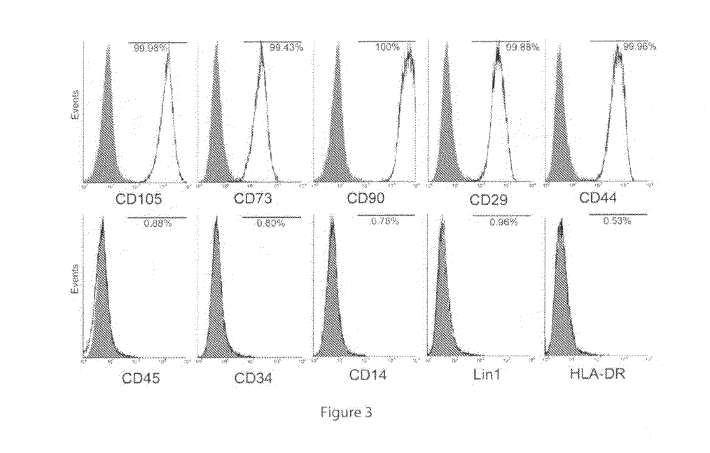

hMSCs were also differentiated into osteoblasts and adipocytes in vitro, and cell phenotype was analyzed by flow cytometry. FIG. 3 shows that the cells express CD105, CD73, CD90 (Thy-1), CD29, and CD44. The cells do not express CD45, CD34, CD14, Lint, and HLA-DR. Therefore, these cells are typical MSCs (Pittenger et al., Science 284: 143-7 [1999]; Dominici et al., Cytotherapy 8: 315-7 [2006]).

More specifically, FIG. 3 shows the phenotype of hMSCs. Flow cytometric analysis was conducted on ex vivo-expanded hMSCs to determine the expression of CD14, CD29, CD34, CD44, CD45, CD73, CD90 (Thy-1), CD105, human lineage cocktail (Lint, i.e. CD3, CD14, CD16, CD19, CD20, and CD56), and HLA-DR. Histograms show the relative intensity of hMSCs for various cell-surface antigens. Numbers indicate the percentage of cells in the population whose staining intensity with the specific antibody (white) was greater than that with the respective isotype control (gray).

Improvement of Survival of Irradiated Mice by Intravenous Administration of MSCs Genetically Modified with ECSOD

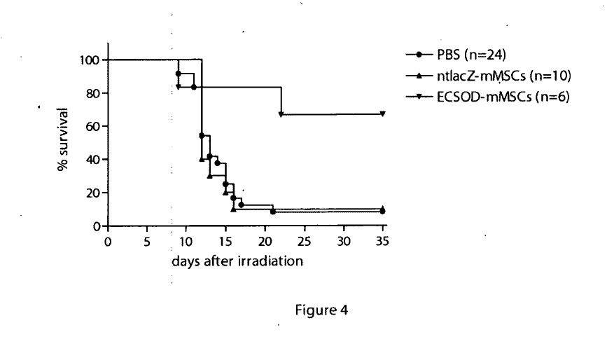

To determine whether intravenous administration of ECSOD gene-modified-MSCs, i.e. ECSOD-MSCs, has a therapeutic effect on radiation damage, 5-week-old female BALB/c mice were given 9 Gy total body γ irradiation from a ¹³⁷Cs source. Twenty-four hours later, the animals were given a tail vein injection of PBS, Ad5CMVntlacZ-transduced mMSCs, or Ad5CMVECSOD-transduced mMSCs. Mouse survival was then monitored daily for 35 days. As shown in FIG. 4, 66.7% of animals in ECSOD gene-modified mMSCs treatment group survived for over 35 days whereas only 10% of animals in ntlacZ gene-modified mMSCs treatment group and 8.3% of animals in PBS treatment group survived more than 35 days. Therefore, intravenous treatment with MSCs genetically modified with ECSOD after whole-body radiation exposure improves survival.

More specifically, FIG. 4 is a Kaplan-Meier survival curve showing that intravenous treatment with ECSOD gene-modified mMSCs improves survival of irradiated mice. Five-week-old, female BALB/c mice were given 9 Gy total body γ irradiation from a ¹³⁷Cs source. Twenty-four hours later, the animals were given a tail vein injection of PBS, ntlacZ gene-modified mMSCs (ntlacZ-mMSCs), or ECSOD gene-modified mMSCs (ECSOD-mMSCs). Mouse survival was monitored every day for 35 days. A Kaplan-Meier survival curve was used for data analysis and statistical significance was determined between groups using one-way analysis of variance (ANOVA) followed by post hoc analysis with Tukey test. P<0.05 was considered statistically significant. P>0.05 for ▴ vs. and P<0.01 for ▾ vs. or ▴.

### Removal of Cell Clumps in MSC Suspension by Filtration Method

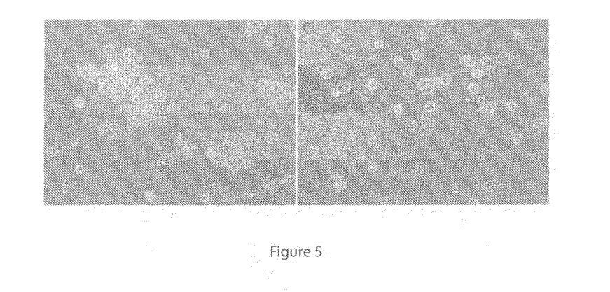

To determine whether filtration method can remove cell clumps in mesenchymal stem cell (MSC) suspension, mMSCs were suspended in PBS at a concentration of 2.5×10⁶ cells/ml. Under a phase contrast microscope, many cell clumps were observed (FIG. 5A). The cells were then filtered through a cell strainer with 40 μm nylon mesh to remove cell clumps. Under a phase contrast microscope, no cell clumps were observed (FIG. 5B). Therefore, filtration of MSCs can remove cell clumps in MSC suspension.

More specifically, FIGS. 5A and 5B show images depicting the removal of cell clumps in mouse mesenchymal stem cell (mMSC) suspension by filtration method. mMSCs were suspended in phosphate buffered saline (PBS) at a concentration of 2.5×10⁶ cells/ml. The cells were then filtered through a cell strainer with 40 μm nylon mesh to remove cell clumps. FIG. 5A is a photomicrograph showing mMSCs before filtration. FIG. 5B is a photomicrograph showing mMSCs after filtration with original magnification: ×20.

### Ionizing Radiation Causes Bone Marrow Failure in Mice.

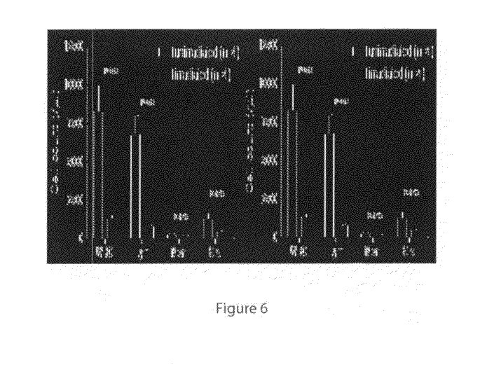

To investigate whether radiation induces bone marrow failure in mice, five-week-old female BALB/c mice were given 9 Gy total body γ irradiation from a ¹³⁷Cs source at a dose rate of 1.23 Gy/min. Seven days later, the mice were euthanized and peripheral blood and bone marrow were analyzed. Complete blood count (CBC) showed that white blood cell and lymphocyte counts in peripheral blood of the irradiated mice decreased (FIG. 6). The number of nucleated bone marrow cells of the femur of irradiated mice also decreased. Furthermore, necropsy of mice at 13-17 days after 9 Gy total body ¹³⁷Cs γ irradiation revealed widespread bleeding in most internal organs.

More specifically, FIG. 6 shows the effect of ¹³⁷Cs γ radiation on cell counts of peripheral blood in mice. Five-week-old female BALB/c mice were given 9 Gy total body γ irradiation from a ¹³⁷Cs source. Seven days later, the mice were sacrificed. Peripheral blood was analyzed for complete blood count (CBC) using the fully automated instrument VetScan HM2 Hematology System (Abaxis Inc., Union city, CA). The counts of white blood cell (WBC) and lymphocytes (Lym) in whole blood were then compared with those of un-irradiated mice. The data was expressed as mean±SEM (n=4) and statistically analyzed using at test.

Intravenous Administration of MSCs Genetically Modified with ECSOD Improves Survival in Irradiated Mice

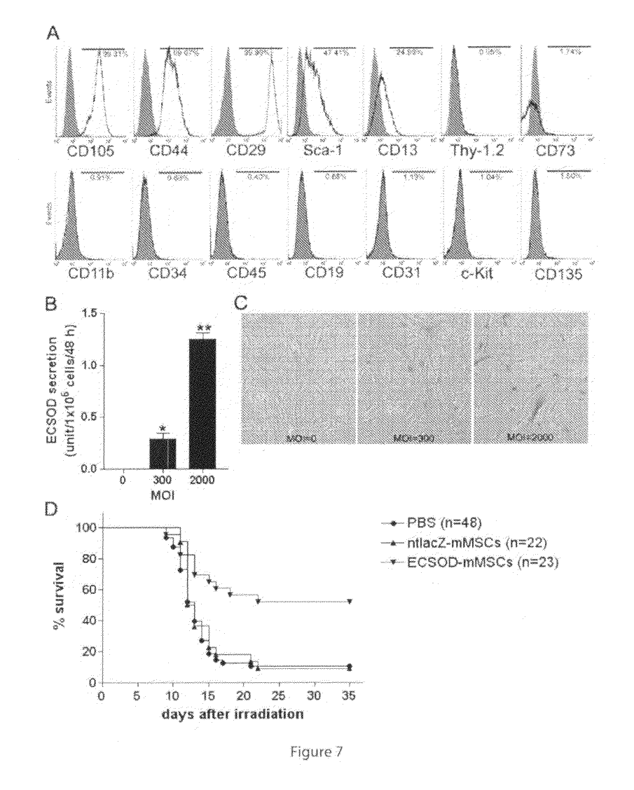

To test the hypothesis that MSCs genetically modified with ECSOD have a radioprotective effect, mouse MSCs (mMSCs) were isolated by their adherence to tissue-culture plastic from six-week-old female BALB/c mice and ex vivo expanded as previously described (Sun et al., Stem Cells. 21:527-35 [2003]; Peister et al. Blood 103:1662-8 [2004]; Abdel-Mageed et al., Blood 113: 1201-3 [2009]). The cells were differentiated into osteoblasts and adipocytes in vitro, and cell phenotype was analyzed by flow cytometry. FIG. 7A shows that the cells express CD105, CD44, CD29, stem cell antigen-1 (Sca-1), and CD13. The cells do not express CD11b, CD34, CD45, CD19, CD31, CD117 (c-Kit), CD135, CD90 (Thy-1.2), or CD73. Therefore, these cells are typical MSCs.

To study the efficacy of adenoviral gene transfer in mMSCs, mMSCs were transduced with Ad5CMVECSOD, an adenovirus carrying human ECSOD gene under the control of cytomegalovirus (CMV) promoter (Chu et al. Circ Res. 92:461-8 [2003]), and culture supernatant was analyzed for superoxide dismutase (SOD) activity. FIG. 7B shows a dose-dependent secretion of biologically active ECSOD by Ad5CMVECSOD-transduced mMSCs. mMSCs were further transduced with Ad5CMVntlacZ, an adenovirus carrying nuclear-targeted β-galactosidase gene ntlacZ under the control of CMV promoter (Chu et al. Circ Res. 92:461-8 [2003]), and analyzed by X-gal staining. As shown in FIG. 7C, transduction efficiency is dose-dependent.

To determine whether intravenous administration of mMSCs genetically modified with ECSOD has a therapeutic effect for radiation damage, five-week-old female BALB/c mice were given 9 Gy total body γ irradiation from a ¹³⁷Cs source at a dose rate of 1.23 Gy/min. Twenty-four hours later, the animals were given a tail vein injection of phosphate-buffered saline (PBS), Ad5CMVntlacZ-transduced mMSCs, or Ad5CMVECSOD-transduced mMSCs. As shown in FIG. 7D, 52% of animals in ECSOD gene-modified mMSCs treatment group survived for 35 days, whereas only 9% of animals in ntlacZ gene-modified mMSCs treatment group and 10% of animals in PBS treatment group survived for 35 days. Furthermore, all mice that survived for 35 days also survived for five months. These findings demonstrate for the first time that intravenous administration of MSCs genetically modified with ECSOD improves survival in irradiated mice, highlighting its clinical potential for the treatment of radiation injury resulting from a radiation accident, nuclear accident, nuclear terrorism, nuclear war, other radiological emergencies, space travel, radiation therapy, and diagnostic radiology.

More specifically, FIG. 7 shows the radioprotective effect of mesenchymal stem cells genetically modified to secrete extracellular superoxide dismutase. FIG. 7A shows the phenotype of mMSCs. Flow cytometric analysis was conducted on ex vivo-expanded mMSCs to determine the expression of CD11b, CD13, CD19, CD29, CD31, CD34, CD44, CD45, CD73, CD90 (Thy-1.2), CD105, CD117 (c-Kit), CD135, and Sca-1. Histograms show the relative intensity of mMSCs for various cell-surface antigens. Numbers indicate the percentage of cells in the population whose staining intensity with the specific antibody (white) was greater than that with the respective isotype control (gray). FIG. 7B shows the secretion of biologically active ECSOD by Ad5CMVECSOD-transduced mMSCs. mMSCs were transduced with Ad5CMVECSOD at multiplicity of infections (MOI, defined as plaque-forming units/cell) of 0, 300, or 2,000 for 48 hours, the virus-containing culture medium was removed, and the cells were washed three times with PBS and further incubated in fresh culture medium for 48 hours. The culture supernatant was collected and analyzed for SOD activity using a SOD activity assay kit (Cayman Chemical Company, Ann Arbor, Mich.). The data was expressed as mean±SEM (n=3) and analyzed statistically using a one-way analysis of variance (ANOVA) followed by post-hoc analysis with a Tukey test. *P<0.05 versus MOI 0; **P<0.05 versus MOI 0 or 300. FIG. 7C includes photomicrographs showing expression of nuclear-targeted β-galactosidase by Ad5CMVntlacZ-transduced mMSCs. mMSCs were transduced with Ad5CMVntlacZ at MOI 0, 300, or 2,000 for 48 hours. The cells were X-gal stained for β-galactosidase activity and the blue nuclear-targeted β-galactosidase-positive Ad5CMVntlacZ-transduced mMSCs were identified. Original magnification ×40. FIG. 7D shows that improvement in survival of irradiated mice by mesenchymal stem cells genetically modified with extracellular superoxide dismutase. Five-week-old female BALB/c mice were given 9 Gy total body γ irradiation from a ¹³⁷Cs source (Gammacell 1000; MDS Nordion, Ottawa, ON) at a dose rate of 1.23 Gy/min. Twenty-four hours later, the animals were given a tail vein injection of 200 μl PBS, 0.5×10⁶ ntlacZ gene-modified mMSCs (ntlacZ-mMSCs, MOI=2,000) in 200 μl PBS, or 0.5×10⁶ ECSOD gene-modified mMSCs (ECSOD-mMSCs, MOI=2,000) in 200 μl PBS. Mouse survival was then monitored for 35 days. Kaplan-Meier survival curve was used for data analysis, and statistical significance was determined using log-rank test and one-way ANOVA followed by post-hoc analysis with Tukey test. P<0.05 was considered statistically significant. The difference between the three groups was statistically significant by log rank test (P=0.002) and ANOVA (P<0.001). Furthermore, P<0.001 for ECSOD-mMSCs versus PBS, P<0.001 for ECSOD-mMSCs versus ntlacZ-mMSCs, and P>0.05 for ntlacZ-mMSCs versus PBS by a Tukey test. In this study, four separate experiments were conducted and the result of each experiment was similar.

Mice given 9 to 10 Gy total body irradiation die a hematologic death 10 to 14 days after exposure (Millar et al., Int J Radiat Oncol Biol Phys. 8: 581-3 [1982]). It has been found that MSCs migrate to radiation-injured tissues, such as bone marrow and gut after intravenous administration (Chapel et al., J Gene Med. 5: 1028-38 [2003]). Therefore, the improvement in survival of irradiated mice may result from the scavenger of O₂⁻ in the irradiated tissues such as bone marrow and gastrointestinal tract by ECSOD secreted from Ad5CMVECSOD-transduced MSCs.

### Persistence of Adenoviral-Mediated Transgene Expression In Vitro

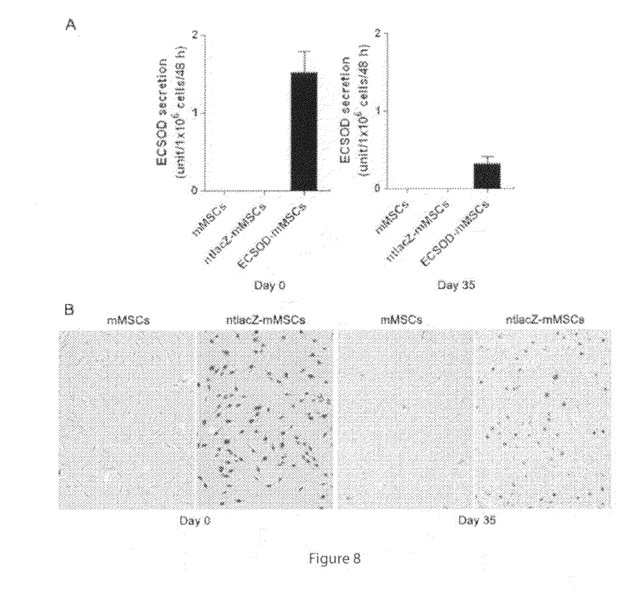

To study the persistence of ECSOD transgene expression in vitro, mMSCs were transduced with Ad5CMVECSOD at MOI 2,000 for 48 hours. The virus-containing culture medium was removed, and the cells were washed 3 times with PBS. The cells were then counted, cultured in fresh culture medium for 48 hours, and culture supernatant was collected. The cells were further incubated in fresh culture medium, and culture medium was changed every 2-3 days until day 35. The cells were then cultured in fresh culture medium for 48 hours, and culture supernatant was collected. The 48-hour culture supernatant at day 0 and 35 after transduction were then analyzed for ECSOD secretion using a SOD activity assay kit (Cayman Chemical Company). As shown in FIG. 8A, Ad5CMVECSOD-transduced mMSCs secreted 1.52±0.27 (mean±SEM, n=3) biologically active ECSOD at day 0 and 0.32±0.09 (mean±SEM, n=3) biologically active ECSOD at day 35. Therefore, Ad5CMVECSOD-transduced mMSCs still secreted relatively high amount of biologically active ECSOD even at day 35.

To study the persistence of ntlacZ transgene expression in vitro, mMSCs were transduced with Ad5CMVntlacZ at MOI 2,000 for 48 hours. The virus-containing culture medium was removed, and the cells were washed 3 times with PBS. Some cells were X-gal stained for β-galactosidase activity and the blue nuclear-targeted β-galactosidase positive Ad5CMVntlacZ-transduced mMSCs were identified. Other cells were further incubated in fresh culture medium, and the culture medium was changed every 2-3 days until day 35. The cells were then X-gal stained for β-galactosidase activity and the blue nuclear-targeted β-galactosidase positive Ad5CMVntlacZ-transduced mMSCs were identified. As shown in FIG. 8B, the percentage of cells expressing β-galactosidase was 99%±1 (mean±SEM, n=3) at day 0 and 24%±5 (mean±SEM, n=3) at day 35 after transduction. Therefore, the percentage of cells expressing β-galactosidase was still high even at day 35.

More specifically, FIG. 8 shows that persistence of adenoviral-mediated transgene expression in vitro. FIG. 8A shows secretion of biologically active ECSOD by Ad5CMVECSOD-transduced mMSCs at various time intervals after transduction. mMSCs were transduced with Ad5CMVECSOD at multiplicity of infection (MOI, defined as plaque-forming unit/cell) 2,000 for 48 hours. The virus-containing culture medium was removed, and the cells were washed with phosphate-buffered saline (PBS) three times and counted. The cells were cultured in fresh culture medium for 48 hours, and the culture supernatant was collected. The cells were further incubated in fresh culture medium, and the culture medium was changed every 2-3 days until day 35. The cells were counted and then cultured in fresh culture medium for 48 hours, and the culture supernatant was collected. The 48-hour culture supernatant at day 0 and 35 after transduction were analyzed for superoxide dismutase (SOD) activity using a SOD activity assay kit (Cayman Chemical Company, Ann Arbor, Mich.) and ECSOD secretion was then determined. Each value represents mean±SEM (n=3). FIG. 8B contains photomicrographs showing expression of nuclear-targeted β-galactosidase by Ad5CMVnttacZ-transduced mMSCs at various time intervals after transduction. mMSCs were transduced with Ad5CMVntlacZ at MOI 2,000 for 48 hours. The virus-containing culture medium was removed, and the cells were washed with PBS three times. Some cells were X-gal stained for β-galactosidase activity and the blue nuclear-targeted β-galactosidase positive Ad5CMVntlacZ-transduced mMSCs were identified. Other cells were further incubated in fresh culture medium, and the culture medium was changed every 2-3 days until day 35. The cells were then X-gal stained for β-galactosidase activity and the blue nuclear-targeted β-galactosidase positive Ad5CMVntlacZ-transduced mMSCs were identified. The ntlacZ transgene expression at day 0 and 35 after transduction were then determined. Original magnification ×40.

### Effect of ECSOD-MSCs Treatment on Mouse Body Weight Change

The study further shows that ionizing radiation causes body weight change in mice. The post-treatment procedure is that all of the mice are monitored daily and their body weights are recorded daily for 35 days. As each animal presents criteria for euthanasia, it will be euthanized by cardiac terminal puncture. According to standard vivarium practice, the following symptoms are considered cause for euthanasia: tumor size of 2500 cubic millimeter or greater; 20 percent loss of body weight in one week; inability to eat or drink; behavior abnormality; slow, shallow, labored breathing; hunched posture; ruffled fur (for 3 days), failure to groom; hypo- or hyper-thermia; diarrhea or constipation (3 days); skin sores, infections, necrotic tissues and tumors; lethargy (for 3 days); impaired mobility; persistent bleeding; paralysis; CNS signs (persistent seizures, spasticity, weakness); and self-segregation from other animals.

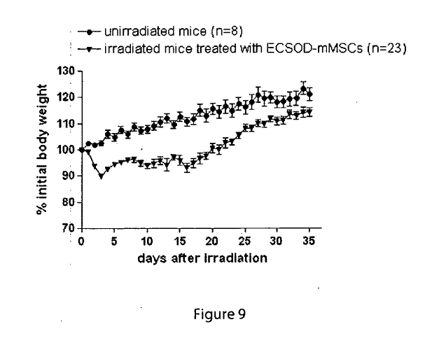

In the study, the most common symptom is the 20% loss of body weight, followed by slow, shallow, labored breathing, hunched posture, ruffled fur, failure to groom (goes along with hunched posture), lethargy, self segregation (goes along with lethargy), and inability to eat or drink. The surviving mice did not have the symptoms mentioned above. The animals were healthy and started to gain weight around 16 days after irradiation. FIG. 9 shows the data of 35 days' body weight change of irradiated mice treated with ECSOD gene-modified mMSCs.

More specifically, FIG. 9 shows the effect of intravenous administration of ECSOD-mMSCs on body weight loss in mice. Five week old female BALB/c mice were given 9 Gy total body γ irradiation from a ¹³⁷Cs source at a dose rate of 1.23 Gy/min. After 24 hours, the animals were given a tail vein injection of 0.5×10⁶ ECSOD-mMSCs. Mouse body weight was then monitored daily for 35 days. Each value represented mean±SEM. Five-week old, female BALB/c healthy unirradiated mice were used as control.

### Effect of Radiation Dose on Mouse Body Weight Change and Survival

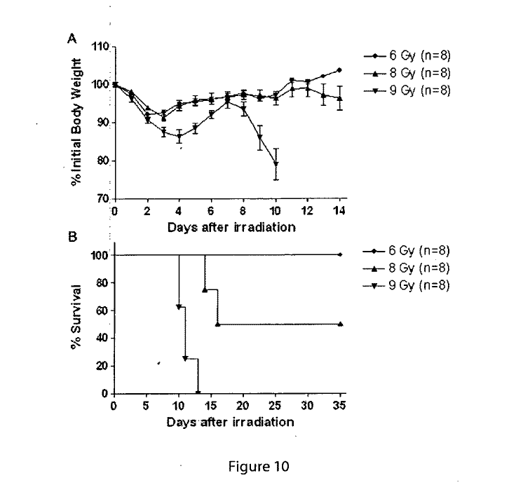

To study the effect of radiation dose on mouse body weight change and survival, mice were exposed to 6, 8, or 9 Gy ¹³⁷Cs γ irradiation. The mice were then monitored daily for 35 days. As shown in FIG. 10, mouse body weight loss and survival were correlated to radiation dose.

More specifically, FIG. 10 shows the effects of irradiation dose on body weight loss and survival in mice. Five-week old female BALB/c mice were given 6, 8, or 9 Gy total body γ irradiation from a ¹³⁷Cs source at a dose rate of 1.23 Gy/min. Mouse body weight loss (FIG. 10A) and survival (FIG. 10B) were monitored daily for 35 days. Each value represents mean±SEM (n=8). A Kaplan-Meier survival curve was used for data analysis.

### Radioprotective Effect of Mesenchymal Stem Cells Genetically Modified to Secrete Extracellular Superoxide Dismutase

Exposure to high dose of ionizing radiation leads to death, cataract formation, and tumor development, which can happen weeks to decades after exposure. At present, no approved drugs or therapies are available for the treatment of radiation injuries. Formation of superoxide anion (O₂⁻) after ionizing radiation is a major determinant of radiation injuries. Irradiated tissues release O₂⁻ for days to months after radiation exposure (Greenberger, Pharmacogenomics 7: 1141-5 [2006]). Extracellular superoxide dismutase (ECSOD), a potent antioxidant enzyme catalyzing the dismutation of O₂⁻, can alleviate oxidative stress. Mesenchymal stem cells (MSCs), a subset of adult stem cells from bone marrow, have been found to migrate to radiation injured tissues after intravenous administration (Chapel et al., J Gene Med. 5: 1028-38 [2003]). Therefore, the cells hold promise as vehicles for adult stem cell-based gene therapy of radiation injuries.

To test the hypothesis that MSCs overexpressing ECSOD have a radioprotective effect, mouse MSCs (mMSCs) were transduced with Ad5CMVECSOD, an adenovirus carrying human ECSOD gene, and secretion of high levels of biologically active ECSOD was detected (FIG. 1A). mMSCs were also transduced with Ad5CMVntlacZ, an adenovirus carrying reporter gene ntlacZ, and secretion of biologically active ECSOD was not detected (FIG. 1B). Mice were then given 9 Gy total body γ irradiation and 24 hours later via a tail vein injection of Ad5CMVECSOD-transduced mMSCs (ECSOD-mMSCs), Ad5CMVntlacZ-transduced mMSCs (ntlacZ-mMSCs), or phosphate-buffered saline (PBS). Remarkably, 52% of mice in ECSOD-mMSCs treatment group survived for 35 days, whereas only 9% of mice in ntlacZ-mMSCs treatment group and 10% of mice in PBS treatment group survived for 35 days. The improvement in survival of irradiated mice might result from the scavenging of O₂⁻ in radiation injured tissues such as bone marrow and gastrointestinal tract by ECSOD secreted from ECSOD-mMSCs. This finding demonstrates for the first time that intravenous administration of MSCs genetically modified to secrete ECSOD improves survival in irradiated mice, highlighting its clinical potential for the treatment of potentially lethal complications of acute radiation syndrome (Abdel-Mageed et al., Blood 113: 1201-3 [2009]).

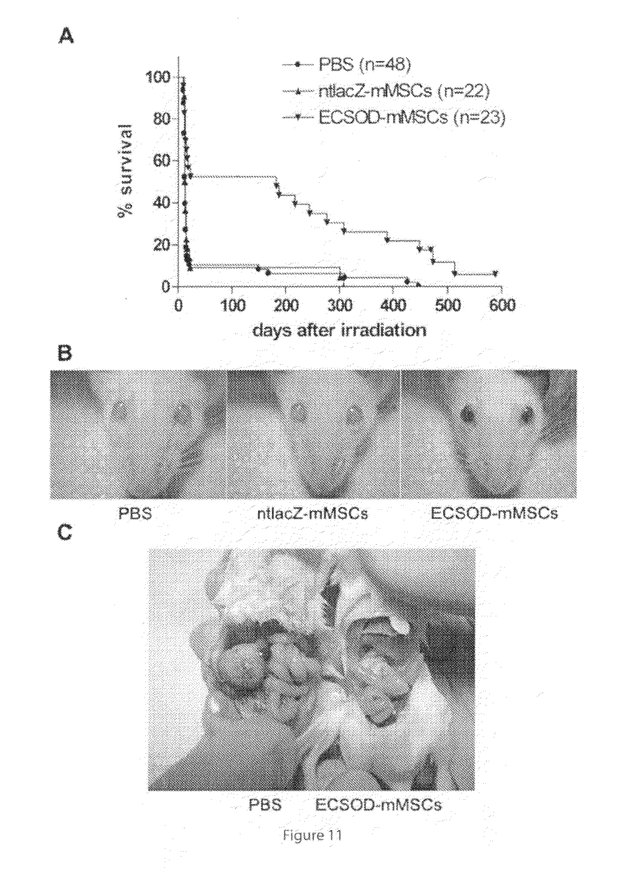

To determine whether intravenous administration of MSCs genetically modified to secrete ECSOD has a beneficial effect in the treatment of delayed radiation effects such as lifespan shortening, cataractogenesis, and carcinogenesis (Epperly et al., Radiat Res. 170: 437-43 [2008]), irradiated mice that had survived for 35 days after PBS, ntlacZ-mMSCs, or ECSOD-mMSCs treatment were monitored for their whole lifespan. Previous studies have demonstrated that overexpression of superoxide dismutase extends lifespan in Drosophila (Orr and Sohal, Science 263: 1128-30 [1994]) and prevents cataract formation in rats (Lin et al., Mol Vis. 11: 853-8 [2005]). In this study, irradiated mice in ECSOD-mMSCs treatment group survived more than 140 days longer than irradiated mice in PBS or ntlacZ-mMSCs treatment group (FIG. 11A). Irradiated mice in ECSOD-mMSCs treatment group developed cataracts 39 days later than irradiated mice in PBS or ntlacZ-mMSCs treatment group (FIG. 11B). No tumor development was observed in irradiated mice in ECSOD-mMSCs treatment group, whereas large abdominal tumor was found in irradiated mice in PBS treatment group (FIG. 11C). Thus, intravenous administration of MSCs genetically modified to secrete ECSOD has extended lifespan, retarded cataract formation, and prevented carcinogenesis in irradiated mice. Therefore, ECSOD-MSCs have a radioprotective effect for both acute radiation syndrome and delayed radiation effects.

More specifically, FIG. 11A-C shows radioprotection by mesenchymal stem cells overexpressing extracellular superoxide dismutase. Mice were given 9 Gy total body γ irradiation and 24 hours later a tail vein injection of PBS, 0.5×10⁶ ntlacZ-mMSCs, or 0.5×10⁶ ECSOD-mMSCs. Mouse survival, cataract formation, and carcinogenesis were monitored over the whole lifespan. FIG. 11A demonstrates that intravenous administration of ECSOD-mMSCs extends lifespan in irradiated mice (P=0.003 by logrank test). FIG. 11B is a photomacrograph showing delay in cataract formation in irradiated mice treated with ECSOD-mMSCs (Picture was taken 172 days after irradiation). FIG. 11C is a photomacrograph showing prevention of carcinogenesis in irradiated mice treated with ECSOD-mMSCs (Picture was taken 449 days after irradiation).

### Example 2

Recently, mouse MSCs (mMSCs) were transduced with adenovirus containing ECSOD and the cells secreted biologically active ECSOD. To ascertain whether Ad5CMVECSOD can infect mMSCs and whether mMSCs genetically modified with ECSOD can secrete functional ECSOD, mMSCs were transduced with Ad5CMVECSOD at MOI 0, 300 or 2,000 for 48 hours. The cells were washed with PBS and further incubated for 48 hours. The culture supernatant was then collected and analyzed for SOD activity. A dose dependent secretion of biologically active ECSOD by Ad5CMVECSOD-transduced mMSCs was confirmed. The efficacy of adenoviral-mediated gene transfer into mMSCs was further examined using the reporting gene ntlacZ. To this end, mMSCs were transduced with Ad5CMVntlacZ at MOI 0, 300, or 2,000. After 48 hours, the expression of nuclear-targeted β-galactosidase in Ad5CMVntlacZ-transduced mMSCs was assessed by X-gal staining. Transduction efficiency of Ad5CMVntlacZ into mMSCs was proven to be dose-dependent. Therefore, adenoviral transduction of mMSCs is effective and mMSCs genetically modified with ECSOD produce and secrete biologically active ECSOD.

In the in vivo study, mesenchymal stem cells were genetically modified with extracellular superoxide dismutase (ECSOD-MSCs) and were given intravenously to improve survival in lethally irradiated mice. To determine whether intravenous administration of ECSOD-MSCs has a therapeutic effect on radiation injury, 5-week-old female BALB/c mice were given 9 Gy total body γ irradiation from a ¹³⁷Cs source. Twenty four hours later, the animals were given a tail vein injection of PBS, Ad5CMVntlacZ-transduced mMSCs, or Ad5CMVECSOD-transduced mMSCs. Mouse survival was then monitored daily for 35 days. Remarkably, 52% of animals in Ad5CMVECSOD-transduced mMSCs treatment group survived for 35 days, whereas only 9% of animals in Ad5CMVntlacZ-transduced mMSCs treatment group and 10% of animals in PBS treatment group survived for 35 days (Abdel-Mageed et al., Blood 113: 1201-3 [2009]). Therefore, intravenous administration of ECSOD-MSCs after whole body radiation exposure improves survival in mice, highlighting its clinical potential for the treatment of ARS.

Animal studies have shown that MSCs can migrate to radiation-injured tissues such as bone marrow and gastrointestinal tract after intravenous administration (Chapel et al, J Gene Med. 5: 1028-38 [2003]; Bensidhoum et al., Blood 103: 3313-9 [2004]; Mouiseddine et al., Br J Radiol. 80 Spec No 1: S49-55 [2007]). In one study, animals received TBI at 8 Gy and then were treated with hematopoietic stem cells and GFP-labeled MSCs. MSCs were found in the injured muscle, skin, bone marrow and gut up to 82 days post-infusion (Chapel et al, J Gene Med. 5: 1028-38 [2003]). Further, MSCs have been shown to be resistant to ionizing radiation and sustain hematopoietic reconstitution after irradiation (Chen et al., Int J Radiat Oncol Biol Phys. 66: 244-53 [2006]; Greenberger et al., Acta Haematol. 96: 1-15 [1996]; Drouet et al., Bone Marrow Transplant 35: 1201-9 [2005]). Therefore, MSCs appear to be good vehicles for adult stem cell-based gene therapy to transport therapeutic gene to the radiation-injured tissue sites (Herodin et al., Folia Histochem Cytobiol. 43: 233-7 [2005]; Li et al., Zhongguo Shi Yan Xue Ye Xue Za Zhi 15: 905-8 [2007]).

### Preliminary Studies

The intestinal epithelium is the most rapidly self-renewing tissue in adult mammals. In murine small intestine, the one-layer epithelium renews every 4-5 days. This vigorous proliferation starts at the crypt bottom and ends at the villus tip. The classical receptor cells are separated by supporting cells with small protrusions on their apical surfaces, the crypt cell is always surrounded by one or two specialized electron-lucent supporting cells which also bear microvillus-like apices. The process is believed to be fueled by intestinal stem cells that may reside near the crypt bottom (Yen and Wright, Stem Cell Rev, 2: 203-12 [2006]; Barker et al., Genes Dev. 22: 1856-64 [2008]). Each crypt is formed of approximately 250 cells, of which about 160 are proliferative. The proliferative cells are arranged as a series of 10 rings. Each ring has about 16 cells and the rings start at the 4^{th} position from the bottom of the crypt and run up to the 14th cell position. The stem cells are believed to be located in the lowermost ring. It is believed that all four types of intestinal epithelial cells (absorptive, enteroendocrine, mucosecreting, and Paneth cells) are derived from a single intestinal stem cell.

For the elusive intestinal stem cells, two hypotheses have been proposed. The “+4 position” model places the putative intestinal stem cells at position +4 relative to the crypt bottom, with the first three positions being occupied by Paneth cells. The +4 cells are actively cycling and thus extremely sensitive to radiation (Potten et al., Cell Tissue Kinet. 10: 557-68 [1977]; Potten et al., Novartis Found Symp. 235: 66-79; discussion 79-84, 101-4 [2001]). The “stem cell zone” model states that crypt base columnar cells, the small, undifferentiated, cycling cells hidden between Paneth cells, may represent the true intestinal stem cells (Bjerknes and Cheng, Gastroenterology 116: 7-14 [1999]). Although the isolation and characterization of putative intestinal stem cells from mouse jejunum were conducted using the Hoechst 33342 positive side population sorting method (Goodell et al., J Exp Med 183: 1797-806 [1996]), the cells could not be cultured for more than two weeks and the fate of the cells could not be determined (Dekaney et al., Gastroenterology 129: 1567-80 [2005]).

It has been suggested that markers such as Musashi-1, Hes-1, β1-integrin, phosphor-PTEN, phosphor-AKT, sFRP5, Sox4, DCAMKL-1, Prominin 1, and Lgr5 can be used to mark intestinal stem cells (Becker et al., Scientific World Journal 8: 1168-76 [2008]; Barker et al., Nature 457:608-11 [2009]; Zhu et al., Nature 457: 603-7 [2009]). Yet, these markers are not unique for intestinal stem cells. Therefore, currently no definitive markers can be used to reliably identify intestinal stem cells and thus self-renewal and differentiation of the intestinal stem cells can not be characterized (Kayahara et al., FEBS Lett. 535: 131-5 [2003]; Barker et al., Genes Dev. 22: 1856-64 [2008]). Moreover, the concept that intestinal epithelial cells derive from crypt intestinal stem cells has recently been challenged. Studies in bone marrow transplant patients demonstrate that donor-derived epithelial cells substantially repopulated the gastrointestinal tract of bone-marrow transplant recipients during epithelial regeneration after graft-versus-host disease (Okamoto et al., Hum Cell. 19: 71-5 [2006]). Donor-derived epithelial cells account for 0.4%-3.6% of small intestine epithelia (Okamoto et al., Nat. Med. 8: 1011-7 [2002]; Okamoto and Watanabe, Dig Dis Sci. 50 Suppl 1:S34-8 [2005]). Therefore, bone marrow-derived cells can differentiate into functional intestinal epithelia and the cells are involved in the regeneration of damaged epithelia in human intestinal tract. Further, donor-derived epithelial cells can be detected eight years after bone marrow transplant. Since intestinal epithelial cell turnover takes only several days, bone marrow-derived cells are believed to be involved in normal intestinal epithelial renewal (Okamoto et al., Nat. Med. 8: 1011-7 [2002]).

This study is designed to determine whether ionizing radiation causes bone marrow failure in mice. To investigate whether radiation induces bone marrow failure in mice, five-week-old female BALB/c mice were given 9 Gy total body γ irradiation from a ¹³⁷Cs source. Seven days later, the mice were euthanized and peripheral blood and bone marrow were analyzed. Complete blood count (CBC) showed that white blood cell and lymphocyte counts in peripheral blood of the irradiated mice decreased (FIG. 6). The number of nucleated bone marrow cells of the femur of irradiated mice also decreased. Furthermore, necropsy of mice at 13-17 days after 9 Gy total body ¹³⁷Cs γ irradiation revealed widespread bleeding in most internal organs (data not shown).

Intravenous Administration of MSCs Genetically Modified with ECSOD Improves Survival in Irradiated Mice

To test the hypothesis that MSCs genetically modified with ECSOD have a radioprotective effect, mouse MSCs (mMSCs) were isolated by their adherence to tissue-culture plastic from six-week-old female BALB/c mice and ex vivo expanded as previously described (Sun et al., Stem Cells. 21:527-35 [2003]; Peister et al. Blood 103:1662-8 [2004]; Abdel-Mageed et al., Blood 113: 1201-3 [2009]). The cells were differentiated into osteoblasts and adipocytes in vitro, and cell phenotype was analyzed by flow cytometry. FIG. 7A shows that the cells express CD105, CD44, CD29, stem cell antigen-1 (Sca-1), and CD13. The cells do not express CD11b, CD34, CD45, CD19, CD31, CD117 (c-Kit), CD135, CD90 (Thy-1.2), or CD73. Therefore, these cells are typical MSCs.

To study the efficacy of adenoviral gene transfer in mMSCs, mMSCs were transduced with Ad5CMVECSOD, an adenovirus carrying human ECSOD gene under the control of cytomegalovirus (CMV) promoter (Chu et al., Circ Res. 92:461-8 [2003]), and culture supernatant was analyzed for superoxide dismutase (SOD) activity. FIG. 7B shows a dose-dependent secretion of biologically active ECSOD by Ad5CMVECSOD-transduced mMSCs. mMSCs were further transduced with Ad5CMVntlacZ, an adenovirus carrying nuclear-targeted β-galactosidase gene ntlacZ under the control of CMV promoter (Chu et al., Circ Res. 92:461-8 [2003]), and analyzed by X-gal staining. As shown in FIG. 7C, transduction efficiency is dose-dependent.

To determine whether intravenous administration of mMSCs genetically modified with ECSOD has a therapeutic effect for radiation damage, five-week-old female BALB/c mice were given 9 Gy total body γ irradiation from a ¹³⁷Cs source at a dose rate of 1.23 Gy/min. Twenty-four hours later, the animals were given a tail vein injection of phosphate-buffered saline (PBS), Ad5CMVntlacZ-transduced mMSCs, or Ad5CMVECSOD-transduced mMSCs. As shown in FIG. 7D, 52% of animals in ECSOD gene-modified mMSCs treatment group survived for 35 days, whereas only 9% of animals in ntlacZ gene-modified mMSCs treatment group and 10% of animals in PBS treatment group survived for 35 days. Furthermore, all mice that survived for 35 days also survived for five months. These findings demonstrate for the first time that intravenous administration of MSCs genetically modified with ECSOD improves survival in irradiated mice, highlighting its clinical potential for the treatment of radiation injury resulting from a radiation accident, nuclear accident, nuclear terrorism, nuclear war, other radiological emergencies, space travel, radiation therapy, and diagnostic radiology.

To study the persistence of ECSOD transgene expression in vitro, mMSCs were transduced with Ad5CMVECSOD at MOI 2,000 for 48 hours. The virus-containing culture medium was removed, and the cells were washed 3 times with PBS. The cells were then counted, cultured in fresh culture medium for 48 hours, and culture supernatant was collected. The cells were further incubated in fresh culture medium, and culture medium was changed every 2-3 days until day 35. The cells were then cultured in fresh culture medium for 48 hours, and culture supernatant was collected. The 48-hour culture supernatant at day 0 and 35 after transduction were then analyzed for ECSOD secretion using a SOD activity assay kit (Cayman Chemical Company). As shown in FIG. 8A, Ad5CMVECSOD-transduced mMSCs secreted 1.52±0.27 (mean±SEM, n=3) biologically active ECSOD at day 0 and 0.32±0.09 (mean±SEM, n=3) biologically active ECSOD at day 35. Therefore, Ad5CMVECSOD-transduced mMSCs still secreted relatively high amount of biologically active ECSOD even at day 35.

To study the persistence of ntlacZ transgene expression in vitro, mMSCs were transduced with Ad5CMVntlacZ at MOI 2,000 for 48 hours. The virus-containing culture medium was removed, and the cells were washed 3 times with PBS. Some cells were X-gal stained for β-galactosidase activity and the blue nuclear-targeted β-galactosidase positive Ad5CMVntlacZ-transduced mMSCs were identified. Other cells were further incubated in fresh culture medium, and the culture medium was changed every 2-3 days until day 35. The cells were then X-gal stained for β-galactosidase activity and the blue nuclear-targeted β-galactosidase positive Ad5CMVntlacZ-transduced mMSCs were identified. As shown in FIG. 8B, the percentage of cells expressing β-galactosidase was 99%±1 (mean±SEM, n=3) at day 0 and 24%±5 (mean±SEM, n=3) at day 35 after transduction. Therefore, the percentage of cells expressing β-galactosidase was still high even at day 35.

Mice given 9 to 10 Gy total body irradiation die a hematologic death 10 to 14 days after exposure (Millar et al., Int J Radiat Oncol Biol Phys. 8: 581-3 [1982]). It has been found that MSCs migrate to radiation-injured tissues, such as bone marrow and gut after intravenous administration (Chapel et al., J Gene Med. 5: 1028-38 [2003]). Therefore, the improvement in survival of irradiated mice may result from the scavenger of O₂⁻ in the irradiated tissues such as bone marrow and gastrointestinal tract by ECSOD secreted from Ad5CMVECSOD-transduced MSCs.

The study further shows that ionizing radiation causes body weight change in mice. The post-treatment procedure is that all of the mice are monitored daily and their body weights are recorded daily for 35 days. As each animal presents criteria for euthanasia, it will be euthanized by cardiac terminal puncture. According to standard vivarium practice, the following symptoms are considered cause for euthanasia: tumor size of 2500 cubic millimeter or greater; 20 percent loss of body weight in one week; inability to eat or drink; behavior abnormality; slow, shallow, labored breathing; hunched posture; ruffled fur (for 3 days), failure to groom; hypo- or hyper-thermia; diarrhea or constipation (3 days); skin sores, infections, necrotic tissues and tumors; lethargy (for 3 days); impaired mobility; persistent bleeding; paralysis; CNS signs (persistent seizures, spasticity, weakness); and self-segregation from other animals.

In the study, the most common symptom is the 20% loss of body weight, followed by slow, shallow, labored breathing, hunched posture, ruffled fur, failure to groom (goes along with hunched posture), lethargy, self segregation (goes along with lethargy), and inability to eat or drink. The surviving mice did not have the symptoms mentioned above. The animals were healthy and started to gain weight around 16 days after irradiation. FIG. 9 shows the data of 35 days' body weight change of irradiated mice treated with ECSOD gene-modified mMSCs.

To study the effect of radiation dose on mouse body weight change and survival, mice were exposed to 6, 8, or 9 Gy ¹³⁷Cs γ irradiation. The mice were then monitored daily for 35 days. As shown in FIG. 10, mouse body weight loss and survival were correlated to radiation dose. At a lethal dose of 9 Gy, mice cannot re-establish their original body weight and all intestinal stem cells might have been killed. Therefore, there is a correlation between the amount of intestinal stem cells and the change of body weight in irradiated mice treated with ECSOD-MSCs. Thus, the “ECSOD-MSCs for radioprotection” approach is a better model organism for intestinal stem cell study.

First, an investigator determined whether intravenous administration of ECSOD-MSCs after total body radiation exposure can enhance the recovery of radiation-injured intestinal stem cells. In order to determine the feasibility of intestinal stem cell study in irradiated mice treated with ECSOD-MSCs, mice were given a total body lethal irradiation and 24 hours later treated with ECSOD-MSCs through tail vein injection. Remarkably, 52% of animals survived for 35 days. The surviving mice started to gain weight around day 16 and eventually reached a normal body weight around day 35 after irradiation (FIG. 9), suggesting a window period of 35 days for the study of intestinal stem cell injury and recovery. These results indicate that the irradiated mice treated with ECSOD-MSCs model organism is beneficial (FIG. 7).

Second, an investigator will determine whether intravenous administration of ECSOD-MSCs after abdominal radiation exposure can enhance the recovery of radiation-injured intestinal stem cells. For abdominal irradiation, mice will receive a selective irradiation to the abdomen using a ⁶⁰Co source. Special care will be taken to avoid irradiation of other body parts by using lead shielding (Mouiseddine et al., Br J Radiol. 80 Spec No 1: S49-55 [2007]). The mice will then be treated with ECSOD-MSCs and intestinal stem cells will be studied.

FIG. 9 shows the effect of intravenous administration of ECSOD-mMSCs on body weight loss in mice. Five week old female BALB/c mice were given 9 Gy total body γ irradiation from a ¹³⁷Cs source at a dose rate of 1.23 Gy/min. After 24 hours, the animals were given a tail vein injection of 0.5×10⁶ ECSOD-mMSCs. Mouse body weight was then monitored daily for 35 days. Each value represented mean±SEM. Five-week old, female BALB/c healthy unirradiated mice were used as control.

FIG. 10 shows the effects of irradiation dose on body weight loss and survival in mice. Five-week old female BALB/c mice were given 6, 8, or 9 Gy total body γ irradiation from a ¹³⁷Cs source at a dose rate of 1.23 Gy/min. Mouse body weight loss (FIG. 10A) and survival (FIG. 10B) were monitored daily for 35 days. Each value represents mean±SEM (n=8). A Kaplan-Meier survival curve was used for data analysis.

### Research Design and Methods

The ECSOD-MSCs for radioprotection approach is a better model organism for studying intestinal stem cells in mice. ECSOD-MSCs treated irradiated mice demonstrate the four following mechanisms of intestinal stem cell regeneration:
1. Enhanced recovery of radiation injured crypt intestinal stem cells may occur. By releasing ECSOD, ECSOD-MSCs can scavenger O₂⁻ in the irradiated intestine to enhance the recovery of injured crypt intestinal stem cells. ECSOD-MSCs also can enhance the recovery of injured crypt intestinal stem cells through paracrine effects. ECSOD-MSCs may fuse with injured crypt intestinal stem cells to enhance tissue repair (Rizvi et al., Proc Natl Acad Sci USA. 103: 6321-5 [2006]).
2. Enhanced recovery of radiation injured bone marrow-derived intestinal stem cells may occur. By releasing ECSOD, ECSOD-MSCs can scavenger O₂⁻ in the irradiated bone marrow to enhance the recovery of injured bone marrow-derived intestinal stem cells. ECSOD-MSCs also can enhance the recovery of injured bone marrow-derived intestinal stern cells through paracrine effects. ECSOD-MSCs may fuse with injured bone marrow-derived intestinal stem cells to enhance tissue repair.
3. Enhanced engraftment of bone marrow-derived intestinal stem cells to intestine may occur. By improving intestinal microenvironment through ECSOD release, paracrine effects, and cell fusion, ECSOD-MSCs can enhance the engraftment of bone marrow-derived intestinal stem cells to radiation injured intestine.
4. Under the influence of radiation injured intestinal microenvironment, engrafted bone marrow-derived cells or even ECSOD-MSCs may trans-differentiate into intestinal stem cells.

It has been demonstrated that MSCs can increase self-renewal of small intestinal epithelium and accelerate structural recovery after radiation injury. In one study, human MSCs were transplanted into SCID mice. Following abdominal irradiation, PCR analysis evidences a low but significant MSCs implantation in small intestine (0.17%). In the presence of MSCs, the small intestinal structure is already recovered at three days after abdominal radiation exposure, whereas untreated mice had partial and transient (three days) mucosal atrophy (Semont et al., Adv Exp Med Biol. 585: 19-30 [2006]). In another study, MSCs from beta-Gal-transgenic mice were transplanted into C57BU6J mice that received abdominal irradiation (13 Gy). At different time points, recipient intestines were examined for the engraftment of donor-derived cells. The result suggested that infused MSCs possess the potency to engraft into irradiated enteric mucosa, but the engraftment rate was too low to produce a therapeutic effect. However, MSCs genetically modified with CXCR4 (the receptor for stromal cell-derived factor-1) engrafted into irradiated intestine at a significantly elevated level and ameliorated intestinal permeability and histopathological damage (Zhang et al., J Biomed Sci. 15: 585-94 [2008]). Therefore, ECSOD-MSCs will have a better chance than native MSCs to engraft in radiation injured intestine, increase self-renewal of in situ intestinal stem cells, and accelerate intestinal structure recovery after radiation injury due to trans-differentiation into intestinal stem cells.

To determine whether intravenous administration of ECSOD-MSCs after total body radiation exposure can enhance the recovery of radiation-injured intestinal stem cells”, intestinal stem cells will be studied every day after irradiation for 35 days. A total body irradiated mice treated with ECSOD-MSCs model organism has been created. In this model organism, both crypt intestinal stem cells and bone marrow-derived intestinal stem cells are injured after irradiation. A window period of 35 days post-irradiation is used for the study of intestinal stem cell injury and recovery.

Five-week-old female BALB/c mice will be given 9 Gy total body γ irradiation from a ¹³⁷Cs source (Gammacell 1000; MDS Nordion, Ottawa, ON) at a dose rate of 1.23 Gy/min as was previously described (Abdel-Mageed et al., Blood 113: 1201-3 [2009]). Twenty-four hours later, the animals will be given a tail vein injection of 200 μl phosphate-buffered saline (PBS), 0.5×10⁶ ntlacZ-MSCS in 200 μl PBS, or 0.5×10⁶ ECSOD-MSCS in 200 μl PBS. Mouse body weight and survival will then be monitored daily for 35 days as shown in FIGS. 9 and 10 above. The same number of mice will be sacrificed daily between day 0 and day 35 after irradiation and the small intestine will be harvested.

Crypt resident intestinal stem cells will be studied at each time interval and compared among the three groups. The amount of crypt resident intestinal stem cells will be compared with the change of mouse body weight to determine whether there is a correlation between them. The crypt resident intestinal stem cells could be crypt intestinal stem cells, bone marrow-derived intestinal stem cells, or both. Since both are radiation injured cells, it is not possible to differentiate them in intestinal crypts by histopathology until intestinal or bone marrow cell specific markers are identified.

Mice of different age (5, 12, and 36 weeks old), sex (female and male), and strain (BALB/c and C57BL/6) will be used. Different radiation dose (6, 8, 9, 10, and 12 Gy) and dose rate (1.23, 3.00, and 5.1 1 Gy/min) will be used. Different time point after radiation exposure (30 minutes, 2 hours, 8 hours, 24 hours, and 2-10 days) and different dose of ECSOD-MSCs (0.1×10⁶, 0.5×10⁶, 1×10⁶, 2×10⁶, and 5×10⁶) will be used for ECSOD-MSCs administration. Single, double, and multiple injections of ECSOD-MSCs will also be conducted. Besides ¹³⁷Cs gamma ray, X-ray, ⁶⁰Co gamma ray, and neutrons may be used in this study.

Furthermore, intraperitoneal injection of ECSOD-MSCs will be conducted so that the cells will not be distributed to other organs such as lung and bone marrow. The result will then be compared with that of intravenous injection of ECSOD-MSCs.

To determine whether intravenous administration of ECSOD-MSCs after abdominal radiation exposure can enhance the recovery of radiation-injured intestinal stem cells, intestinal stem cells will be studied every day after irradiation for a specified number of days.

Although intestinal stem cells are believed to be resident stem cells within the intestine, the possibility of the existence of bone marrow-derived intestinal stem cells can not be excluded. If so, intestinal epithelia can be regenerated in mice treated with ECSOD-MSCs after heavy abdominal irradiation.

The abdominal irradiated mice treated with ECSOD-MSCs model organism was wherein only crypt intestinal stem cells are injured after irradiation. Bone marrow-derived intestinal stem cells are healthy after irradiation. A window period of a specified number of days post-irradiation is used for the study of intestinal stem cell injury and recovery.

At a low radiation dose, crypt intestinal stem cells will be injured and then recover in abdominal irradiated mice treated with ECSOD-MSCs. At a high radiation dose, crypt intestinal stem cells will be severely injured or completely eradicated and will not recover in abdominal irradiated mice treated with ECSOD-MSCs. Yet, the healthy bone marrow-derived intestinal stem cells will migrate to the radiation injured intestine and reconstruct intestinal epithelia.

Five-week-old female BALB/c mice will be given 15 Gy abdominal γ irradiation from a ¹³⁷Cs source (Gammacell 1000; MDS Nordion, Ottawa, ON) at a dose rate of 1.23 Gy/min. Twenty-four hours later, the animals will be given a tail vein injection of 200 μl PBS, 0.5×10⁶ ntlacZ-MSCs in 200 μl PBS, or 0.5×10⁶ ECSOD-MSCS in 200 μl PBS. Mouse body weight and survival will then be monitored daily until they eventually reach a normal body weight around a specified day after irradiation. The same number of mice will be sacrificed daily between day 0 and the specified day after irradiation and the small intestine will be harvested.

Crypt resident intestinal stem cells will be studied at each time interval and compared among the three groups. The amount of crypt resident intestinal stem cells will be compared with the change of mouse body weight to determine whether there is a correlation between them. At a low radiation dose, the crypt resident intestinal stem cells could be crypt intestinal stem cells, bone marrow-derived intestinal stem cells, or both. Since both are radiation injured cells, it is not possible to differentiate them in intestinal crypts by histopathology until intestinal or bone marrow cell specific markers are identified. At a high radiation dose, the crypt resident intestinal stem cells can only be bone marrow-derived intestinal stem cells. These cells will be characterized and their progenitor cells in bone marrow will be identified. These bone marrow-derived intestinal stem cells will be directly isolated from bone marrow, cultured, characterized, and functionally validated in vitro and in vivo.

Mice of different age (5, 12, and 36 weeks old), sex (female and male), and strain (BALB/c and C57BL/6) will be used. Different radiation dose (8, 9, 10, 15, and 30 Gy) and dose rate (1.23, 3.00, and 5.1 1 Gy/min) will be used. Different time point after radiation exposure (30 minutes, 2 hours, 8 hours, 24 hours, and 2-10 days) and different dose of ECSOD-MSCs (0.1×10⁶, 0.5×10⁶, 1×10⁶, 2×10⁶, and 5×10⁶) will be used for ECSOD-MSCs administration. Single, double, and multiple injections of ECSOD-MSCs will also be conducted. Besides ¹³⁷Cs gamma ray, X-ray, ⁶⁰Co gamma ray, and neutrons may be used in this project.

Furthermore, intraperitoneal injection of ECSOD-MSCs will be conducted so that the cells will not be distributed to other organs such as lung and bone marrow. The result will then be compared with that of intravenous injection of ECSOD-MSCs.

To isolate, culture, characterize, functionally validate, and compare stem cell populations from the small intestinal epithelium in vivo and in vitro using irradiated mice treated with ECSOD-MSCs, the following assays will be performed.

Immunohistochemistry will be used to identify intestinal stem cells in small intestine of the mice. Antibodies to murine intestinal stem cell markers such as Lgr5 (leucine-rich-repeat containing G-protein-coupled receptor 5), prominin 1 (PROMI, also called CD133), and DCAMKL-1 (double cortin and CaM kinase-like-1) will be used for standard immunofluorescence staining (Becker et al., Scientific World Journal 8: 1168-76 [2008]; Barker et al., Nature 457: 608-11 [2009]; Zhu et al. Nature 457: 603-7 [2009]; May et al., Stem Cells 26: 630-7 [2008]). Expression of murine stem cell markers in normal small intestine, radiation injured small intestine, and radiation injured small intestine will be compared after ECSOD-MSCs treatment. Histopathology of murine small intestine will also be assessed. Each of the three segments of the small intestine (duodenum, jejunum, and ileum) will be analyzed.

Primary culture of the small intestinal tissue of the mice will be used to isolate crypt intestinal stem cells. The Hoechst 33342 positive side population sorting method will be used to isolate crypt intestinal stem cells from the small intestine of the mice. Flow cytometry sorting and stem cell magnetic selection methods will be used to isolate crypt intestinal stem cells from the small intestine of the mice using antibodies to various intestinal stem cell markers. Laser capture microdissection will be used to isolate mRNA and protein from cells in the putative crypt stem cell zone. Monoclonal and polyclonal antibodies to novel intestinal stem cell markers may be produced to identify true intestinal stem cells. A variety of culture media and growth factors will be tested to culture intestinal stem cells so that an intestinal stem cell specific culture medium can be developed (Zhu et al., Nature 457: 603-7 [2009]). To culture intestinal stem cells, conditioned medium from ECSOD-MSCs, which contains biologically active ECSOD, will be used.

To characterize crypt intestinal stem cells, self-renewal and multi-lineage differentiation will be demonstrated both in vitro and in vivo. Crypt microcolony assay, a measure of intestinal stem cell survival and functional competence, will be performed on the small intestine of irradiated mice treated with ECSOD-MSCs (Withers and Elkind, Int J Radiat Biol Relat Stud Phys Chem Med. 17: 261-7 [1970]). Crypt area will be measured using a Zeiss microscope. Crypt cell apoptotic index will be determined by conventional histological and morphological criteria, terminal deoxynucleotidyl transferase-mediated dUTP-fluorescein nick end labeling (TUNEL) assay, and active caspase-3 expression study (Houchen et al., Am J Physiol Gastrointest Liver Physiol. 279: G858-65 [2000]). BrdUrd labeling index in small intestinal crypts will be used for the crypt survival analysis (Houchen et al., Am J Physiol Gastrointest Liver Physiol. 284: G490-8 [2003]).

To functionally validate the isolated intestinal stem cells, mice will be total body or abdominally irradiated and then injected intravenously or introperitoneally with the isolated intestinal stem cells. The fate of the transplanted cells will be monitored in the recipient mice using donor cell specific markers such as lacZ, GFP, Y chromosome. In these studies, transgenic mice with lacZ or GFP gene will be used for the isolation of intestinal stem cells. The cells will be transplanted into un-transgenic mice. Male mice will be used for isolation of intestinal stem cells. The cells will be transplanted into female mice. The isolated intestinal stem cells may also be transplanted into the wall of the irradiated small intestine by direct injection (Kudo et al., Tohoku J Exp Med. 212: 143-50 [2007]). To enhance the survival of the isolated intestinal stem cells, the irradiated mice may be treated with ECSOD-MSCs and then transplanted with the isolated intestinal stem cells for functional validation.

To compare stem cell populations from the small intestinal epithelium in vivo and in vitro, cell phenotype, epithelial differentiation, crypt regeneration, radiation sensitivity, and cell cycle status analysis will be conducted. This will develop a clonal in vivo assay in irradiated mice treated with ECSOD-MSCs to demonstrate self renewal and multi-lineage differentiation of putative intestinal stem cells.

To create a novel, improved model organism for intestinal stem cell study using the ECSOD-MSCs for radioprotection approach, the following materials and methods will be used.

### Adenoviral Vectors

Two adenoviral vectors will be purchased from University of Iowa Gene Transfer Vector Core (Iowa City, Iowa) and used in the study. Ad5CMVECSOD is a replication-deficient recombinant adenovirus carrying the human extracellular superoxide dismutase (ECSOD) gene under the control of cytomegalovirus (CMV) promoter (Chu et al., Circ Res. 92: 461-8 [2003]). Ad5CMVntlacZ is a replication-deficient recombinant adenovirus carrying the nuclear-targeted β-galactosidase reporter gene ntlacZ under the control of CMV promoter (Chu et al., Circ Res. 92: 461-8 [2003]).

Isolation and Ex Vivo Expansion of Mouse Mesenchymal Stem Cells (mMSCs)

mMSCs will be isolated as previously described (Deng et al., Am J Physiol Cell Physiol 285:C1322-9 [2003]; Sun et al., Stem Cells. 21:527-35 [2003]; Peister et al. Blood 103:1662-8 [2004]; Bivalacqua et al. Am J Physiol Heart Circ Physiol. 292:H1278-90 [2007]; Abdel-Mageed et al., Blood 113: 1201-3 [2009]). Six-week-old female BALB/c mice (The Jackson Laboratory, Bar Harbor, Me.) will be euthanized with CO₂ and femurs and tibias will be removed. Both ends of the bones will be cut and bone marrow will be flushed out using a 18-gauge needle and culture medium for mMSCs [MEM-a (Atlanta Biologicals, Norcross, Ga.); 20% fetal bovine serum (FBS, GIBCO Invitrogen Corp., Carlsbad, Calif.); 100 units/ml penicillin, 100 pg/ml streptomycin, and 250 ng/ml amphotericin B (Atlanta Biologicals); and 2 mM L-glutamine (GIBCO Invitrogen Corp)]. The bone marrow cells will be filtered through a cell strainer with 70-pm nylon mesh (BD Bioscience, Bedford, Mass.), and the cells from each mouse will be plated in a T75 flask (Falcon, Fisher Scientific, Pittsburgh, Pa.). The cells will be incubated at 37° C. with 5% humidified Cog, and mMSCs will be isolated by their adherence to tissue culture plastic. Fresh culture medium will be added and replaced every 2-3 days. The adherent mMSCs will be grown to 90% confluence, harvested with 0.25% trypsin/1 mM EDTA for 2 minutes at 37° C., and diluted 1:3 for ex vivo expansion.

Mice of different age (6, 12, and 36 weeks old) and sex (female and male) will be used for isolation of mMSCs. Transgenic mice with lacZ or GFP gene will also be used for isolation of mMSCs. Besides syngeneic mMSCs from BALB/c mice, allogeneic mMSCs from C57BL/6 mice will be used for this project.

Adenoviral Transduction of mMSCs to Secrete Biologically Active ECSOD

mMSCs will be transduced with adenoviral vectors as previously described (Deng et al., Stem Cells 22: 1279-91 [2004]; Baber et al., Am J Physiol Heart Circ Physiol. 292: H1120-8 [2007]; Abdel-Mageed et al., Blood 113: 1201-3 [2009]). Briefly, mMSCs will be plated at a density of 10,000 cells/cm² in 6-well plates or T75 flasks (Falcon, Fisher Scientific) and incubated overnight. The cells will be counted and then exposed to fresh culture medium containing Ad5CMVECSOD at 0, 300, or 2,000 multiplicities of infection (MOI) for 48 hours. MOI is defined as plaque-forming unit (pfu)/cell. Virus-containing culture medium will be discarded, cells will be washed three times with phosphate buffered saline (PBS), and fresh culture medium will be added. Cells will be counted, cultured for 48 hours, and culture supernatant will be collected. The culture supernatant will then be assayed for the secretion of biologically active ECSOD by Ad5CMVECSOD transduced mMSCs using a SOD activity assay kit (Cayman Chemical Company, Ann Arbor, Mich.).

Adenoviral Transduction of mMSCs to Express β-Galactosidase

mMSCs will be transduced with adenoviral vectors as previously described (Deng et al., Am J Physiol Cell Physiol. 285:C1322-9 [2003]; Deng et al., Life Sci. 78: 1830-8 [2006]; Abdel-Mageed et al., Blood 113: 1201-3 [2009]). Briefly, mMSCs will be plated at a density of 10,000 cells/cm² in 6-well plates or T75 flasks (Falcon, Fisher Scientific) and incubated overnight. The cells will be counted and then exposed to fresh culture medium containing Ad5CMVntlacZ at 0, 300, or 2,000 MOI for 48 hours. To conduct X-gal cytochemistry for β-galactosidase activity, Ad5CMVntlacZ-transduced mMSCs will be washed with PBS, fixed for 5 minutes in fixing solution (2% formaldehyde, 0.2% glutaraldehyde, Sigma, St. Louis, Mo.), washed twice with PBS, and incubated in staining solution (1 mg/ml X-gal, 5 mM K ferricyanide, 5 mM K ferrocyanide, and 2 mM MgC12, Sigma) at 37° C. in the dark overnight. Cells will be washed with PBS and the expression of transgene ntlacZ in Ad5CMVntlacZ-transduced mMSCs will be evaluated by light microscopy scoring of blue cells expressing the nuclear-targeted β-galactosidase activity (Deng et al. Stem Cells 22:1279-91 [2004]; Abdel-Mageed et al., Blood 113: 1201-3 [2009]).

Removal of Cell Clumps in mMSCs Suspension by Filtration Method

mMSCs will be suspended in PBS at a concentration of 2.5×10⁶ cells/ml. The cells will then be filtered through a cell strainer with 40-pm nylon mesh (BD Biosciences, Bedford, Mass.) to remove cell clumps. A phase contrast microscope will be used for the observation of cells before and after filtration.

Intravenous Administration of ECSOD or ntlacZ Gene-Modified mMSCs into Irradiated Mice Through Tail Vein Injection

Five-week-old female BALB/c mice will be given a total body or abdominal irradiation using 9 Gy (or the investigated dose) y irradiation from a ¹³⁷Cs source (Gammacell 1000, Serial Number 122, Model B; MDS Nordion, Ottawa, ON, Canada) at a dose rate of 1.23 Gy/min (or the investigated dose rate). Twenty four hours later (or at the specified time interval), these animals will receive a tail vein injection of 200 μl PBS, 0.5×10⁶ ntlacZ gene-modified mMSCs (ntlacZ-MSCs) in 200 μl PBS, or 0.5×10⁶ ECSOD gene-modified mMSCs (ECSOD-MSCs) in 200 μl PBS. All in vivo experiments will be performed on mice in accordance with institutional and NIH guidelines for the care and use of laboratory animals. To prepare ECSOD or ntlacZ gene modified mMSCs, mMSCs will be transduced with Ad5CMVECSOD or Ad5CMVntlacZ at MOI 2,000 for 48 hours. The virus-containing culture medium will be removed and the cells will be washed three times with PBS. The Ad5CMVECSOD or Ad5CMVntlacZ-transduced mMSCs will then be harvested with 0.25% Trypsin/1 mM EDTA, washed with PBS, and a cell suspension at a concentration of 2.5×10⁶ cells/ml will be prepared in PBS for tail vein injection. For intravenous administration of PBS, Ad5CMVECSOD or Ad5CMVntlacZ-transduced mMSCs into the irradiated mice, 200 μl of PBS or 200 μl of cell suspension will be injected into the tail vein using a 27-gauge needle. A total of 0.5×10⁶ cells (or the specified cell dose) or 200 μl PBS will be injected into each mouse. The mice will then be monitored 35 days for body weight and survival.

### Secretion of ECSOD In Vivo

To detect secretion of ECSOD by ECSOD-mMSCs in vivo, a SOD activity assay kit (Cayman Chemical Company, Ann Arbor, Mich.), which measures all three types of SOD, will be used to measure total SOD activity in mouse peripheral blood, bone marrow, and small intestine samples (Nakane et al., Stroke 32:184-9 [2001]; Bivalacqua et al., J Sex Med. 2:187-97 [2005]). Immunohistochemical staining and Western blot assay using an antibody against human ECSOD (Millipore, Cat. #07-704) will be used to detect production and secretion of ECSOD protein by ECSOD-mMSCs in mouse bone marrow, small intestine, and other tissue samples (Nakane et al., Stroke 32:184-9 [2001]; Choung et al., Exp Dermatol. 13: 691-9 [2004]; Bivalacqua et al., Am J Physiol Heart Circ Physiol. 284: H1408-21 [2003]).

### Complete Blood Count (CBC) Assay

About 200 μl mouse peripheral blood will be analyzed for CBC using the fully automated instrument VetScan HM2 Hematology System (Abaxis Inc., Union city, CA). The counts of white blood cell (WBC), red blood cell (RBC), and platelet in whole blood will then determined.

### Bone Marrow Hematopoietic Stem Cell Assay

Mouse femoral bone marrow will be harvested and the total nucleated cell count will be assessed following erythrocyte lysis. Bone marrow clonogenicity [BFU-E (burst-forming unit-erythroid), CFU-GM (colony-forming unit-granulocyte/macrophage), and CFU-GEMM (colony-forming unit-granulocyte, erythroid, monocyte, and macrophage)] will be evaluated using a short-term assay in the Methylcellulose-Methocult Media (Stemcell Technologies, Vancouver, Canada) (Herodin et al., Exp Hematol. 35(4 Supple 1): 28-33 [2007]). Flow cytometry will be used for CD34+ hematopoietic stem cell counting and apoptosis analysis. Histopathology analysis for bone marrow cells will also be conducted (Fliedner, Curr Opin Hematol. 13: 436-44 [2006]; Zenk, Expert Opin Investig Drugs. 16: 767-70 [2007]). Cytokines in bone marrow will also be analyzed (Chao, Exp Hematol. 35(4 Suppl 1): 24-7 [2007]). Whole bone marrow will further be used for MSCs CFU assay (Sun et al., Stem Cells. 21:527-35 [2003]; Peister et al. Blood 103:1662-8 [2004]). RNA microarray analysis will be performed to investigate up or down regulation of the genes responsible for apoptosis.

Kinetics of Transplanted ECSOD Gene-Modified mMSCs in Irradiated Mice

mMSCs will be isolated from transgenic mice with lacZ or GFP gene and then transduced with Ad5CMVECSOD. The cells will be used for the treatment of irradiated un-transgenic mice. Flow cytometry analysis for green cells in bone marrow and histology analysis for green (GFP+) or blue (lacZ+) cells in bone marrow and small intestine will be conducted. Double-immunostaining was used for GFP or lacZ and other markers for cell differentiation analysis. Furthermore, mMSCs isolated from male mice will be transduced with Ad5CMVECSOD and then transplanted into irradiated female mice. The Y chromosome will then be used for cell fate analysis.

### Data Analyses

Data will be expressed as mean±SEM and analyzed statistically using a t-test or a one-way analysis of variance (ANOVA) followed by post hoc analysis with a Tukey test. A Kaplan-Meier survival curve will be used for mouse survival data analysis. For each experiment, PBS, ntlacZ gene-modified mMSCs will be used as controls for ECSOD gene-modified mMSCs. Unmodified mMSCs may also be used as a control.

Filtration of mMSCs to remove cell clumps before tail vein injection can reduce mortality in mice (Hou et al., Proc Natl Acad Sci USA 96: 7294-9 [1999]; Anjos-Afonso et al, J Cell Sci. 117: 5655-64 [2004]; Von Liittichau et al., Stem Cells Dev. 14: 329-36 [2005]). In one study, 2×10⁵ to 14×10⁶ mMSCs were passed through a 40-μm sterile filter before the cells were transplanted by tail vein injection (Hou et al., Proc Natl Acad Sci USA 96: 7294-9 [1999]). Further, in human bone marrow transplantation setting, the bone marrow collected from the donor is routinely filtered through the “Baxter Fenwal Bone Marrow Collection Container with Flexible Pre-Filter” before it is transplanted to the recipient. This container has two filters: one is 500 μm and the other is 200 μm. Filtration of mMSCs to remove cell clumps before tail vein injection will prevent mortality.

To confirm this, the following in vitro study was performed. mMSCs were suspended in PBS at a concentration of 2.5×10⁶ cells/ml. Under a phase contrast microscope, many cell clumps were observed (FIG. 7A). The cells were filtered through a cell strainer with 40 μm nylon mesh (BD Biosciences, Bedford, Mass.) to remove cell clumps. Under a phase contrast microscope, no cell clumps were observed (FIG. 7B). Therefore, filtration of mMSCs through a 40 μm nylon mesh successfully removed cell clumps that would cause mouse death after tail vein injection.

To verify that filtration of mMSCs to remove cell clumps before tail vein injection can reduce mouse mortality; a pilot in vivo study was conducted. In this study, 15 five-week-old female BALB/c mice were given 9 Gy total body γ irradiation from a ¹³⁷Cs source (Gammacell 1000, MDS Nordion, Ottawa, ON) at a dose rate of 1.23 Gy/min. Twenty-four hours later, these animals were divided into three groups of five mice each and given a tail vein injection of 0.1×10⁶, 0.5×10⁶, or 1×10⁶ filtered mMSCs in 200 μl PBS. No signs of distress were observed during and following injection. The mice were closely monitored and no animals died within seven days after mMSCs tail vein injection. Filtered Ad5CMVntlacZ or Ad5CMVECSOD-transduced mMSCs were used in the following experiment and no mice died within seven days after ntlacZ-MSCs or ECSOD-MSCs tail vein injection. Therefore, it was confirmed that filtration of mMSCs before tail vein injection can prevent the death of animals due to pulmonary embolism. Filtered ECSOD-MSCs were used for this proposed research project.

FIG. 5 includes photomicrographs showing the removal of cell clumps in mouse mesenchymal stem cell (mMSC) suspension after filtration through a 40 μm nylon mesh. mMSCs were suspended in phosphate buffered saline (PBS) at a concentration of 2.5×10⁶ cells/ml. The cells were then filtered through a cell strainer with 40 μm nylon mesh (BD Biosciences, Bedford, Mass.) to remove cell dumps. FIG. 5A is a photomicrograph showing mMSCs before filtration. FIG. 5B is a photomicrograph showing mMSCs after filtration. Magnification: ×20.

### Variance in Radiation Sensitivity Among Mice

The allocation of mice was randomized from different litters to the various arms to avoid this potentially negative effect.

### 35 Day Observation Period is Too Short for Mouse Survival Assay

In the pilot study, all of the mice that survived for 35 days lived for over five months. Therefore, the 35 day observation period is appropriate.

Syngeneic mMSCs Versus Allogeneic mMSCs

Besides the syngeneic mMSCs isolated from BALB/c mice (H-2^{d}), allogeneic mMSCs isolated from C57BL/6 mice (H-2^{b}) will be used in the study. The fate of syngeneic and allogeneic ECSOD gene-modified mMSCs in irradiated BALB/c mice will then be investigated. The result will be helpful in solving the controversy that MSCs may or may not be used as “universal cells” for allogeneic transplantation without donor-recipient human leukocyte antigen (HLA) matching (Barry and Murphy, Int J Biochem Cell Biol. 36: 568-84 [2004]; Taupin, Curr Opin Investig Drugs. 7: 473-81 [2006]; Eliopoulos et al., Blood 106: 4057-65 [2005]; Sudres et al., J Immunol. 176: 7761-7 [2006]).

### Fresh Versus Frozen ECSOD-MSCs

Besides freshly prepared ECSOD-MSCs, frozen ECSOD-MSCs will be used to treat irradiated mice. For this study, mMSCs will be transduced with Ad5CMVECSOD at MOI 2,000 for 48 hours and then the cells in will be stored in liquid nitrogen for months or years. The frozen ECSOD-MSCs will be thawed and immediately used for the treatment of irradiated mice. The efficacy of frozen ECSOD-mMSCs will then be assessed.

Other Gene Transfer Vectors to Genetically Modify mMSCs

Besides adenovirus, other gene transfer vectors, such as plasmid, will be used to genetically modify mMSCs for the production and secretion of ECSOD. In the study, a very high MOI (300-2,000) of adenovirus was used to transduce mMSCs in order to see a high transduction efficiency and high level of ECSOD secretion. In the future, a plasmid will be constructed containing the ECSOD gene and transfect mMSCs with the plasmid for high level of ECSOD secretion (Epperly et al., Radiat Res. 170: 437-43 [2008]). Irradiated mice will be treated with mMSCs transfected with the plasmid containing ECSOD.

The “ECSOD-MSCs for radioprotection approach can be used to enhance the recovery of both injured crypt intestinal stem cells and injured endothelial cells, and provides a better chance to isolate intestinal stem cells in irradiated mice treated with ECSOD.

### Alternative Methods and Approaches

Circadian Rhythm of Intestinal Stem Cells.

Mice will be irradiated at 2:00 p.m. for all experiments. Mice may also be irradiated at the time of peak stem cell DNA synthetic activity, which is 3:00 a.m. for the small intestine (Potten et al., Cell Tissue Kinet. 10: 557-68 [1977]). This can be achieved using a reverse light cycle room where mice are acclimatized for two weeks prior to use (Booth et al., Int J. Cancer. 86: 53-9 [2000]).

Intra-Bone Marrow Injection of ECSOD Gene-Modified mMSCs

Intra-bone marrow injection, i.e. intra-femoral or intra-tibial injection, is a newly established strategy for bone marrow stem cell transplantation (Zhang et al., Stem Cells. 22; 1256-62 [2004]; Ikehara, Ann N.Y. Acad Sci. 1051: 626-34 [2005]; Eguchi et al., Transplant Proc. 40: 574-7 [2008]). Intra-bone marrow injection of ECSOD gene-modified mMSCs may be used in the study.

Intravenous or intraperitoneal administration of ECSOD-mMSCs prior to radiation exposure for prophylactic treatment of radiation injury to intestinal stem cells may be used to examine the effect of ECSOD gene-modified mMSCs on protecting intestinal stem cells against radiation induced cell death (Greenberger, Pharmacogenomics 7: 1141-5 [2006]).

The above description is that of the current embodiment of the invention. Various alterations and changes can be made without departing from the spirit and broader aspects of the invention as defined in the appended claims, which are to be interpreted in accordance with the principles of patent law including the doctrine of equivalents. Any reference to claim elements in the singular, for example, using the articles “a,” “an,” “the” or “said,” is not to be construed as limiting the element to the singular.

The embodiments of the invention in which an exclusive property or privilege is claimed are defined as follows:

1. A method of treating or preventing radiation damage by:
administering to a patient in need of treatment at least one therapeutically effective amount of a mesenchymal stem cell genetically altered to secrete extracellular superoxide dismutase.

2. The method of claim 1, wherein said administering step includes administering in a manner selected from the group consisting of intravenous administration, intra-bone marrow administration, intra-arterial administration, intra-cardiac injection, intracerebral injection, intraspinal injection, intra-peritoneal injection, intra-muscular injection, subcutaneous injection, parenteral administration, intra-rectal administration, intra-tracheal injection, intra-nasal administration, intradermal injection, intra-intestinal, and combinations thereof.

3. The method of claim 1, wherein said administering step includes administering the mesenchymal stem cells at a location selected from the group consisting of systemically, at the site of injury, at an adjacent site to the site of injury, and at a site remote from the site of injury, wherein the mesenchymal stem cells migrate to the site of injury after administration.

4. The method of claim 1, wherein said administering step includes administering to a patient in need of treatment multiple therapeutically effective amounts of the mesenchymal stem cells.

5. The method of claim 1, further including genetically modifying the mesenchymal stem cells with a vector to enable the mesenchymal stem cells to secrete extracellular superoxide dismutase.

6. A therapeutic for treating and/or preventing radiation damage, said therapeutic comprising:
genetically modified mesenchymal stem cells capable of secreting extracellular superoxide dismutase.

7. The therapeutic of claim 6, wherein said mesenchymal stem cells are genetically modified using an appropriate viral vector selected from the group consisting of an adenoviral vector, a retroviral vector, a lentiviral vector, and a plasmid to transfer cDNA of extracellular superoxide dismutase into said mesenchymal stem cells for production and secretion of extracellular superoxide dismutase.

8. The therapeutic of claim 6, wherein said mesenchymal stem cells are isolated from locations selected from bone marrow, umbilical cord blood, adipose tissue, and peripheral blood.

9. The therapeutic of claim 6, wherein said mesenchymal stem cells are selected from the group consisting of autologous, allogeneic, syngeneic, and xenogeneic mesenchymal stem cells, with respect to a patient receiving said therapy.

10. The therapeutic of claim 6 for use in treating damage created by radiation or other agents having similar mechanisms of action.

11. The therapeutic of claim 10, wherein said radiation damage is damage selected from the group consisting of cell injury, tissue damage, organ dysfunction, acute radiation syndrome, delayed radiation effects including radiation-induced lifespan shortening, cataractogenesis, and carcinogenesis.

12. The therapeutic of claim 6 for use in preventing damage created by radiation or other agents having similar mechanisms of action.

13. The therapeutic of claim 12, wherein said therapy is administered prior to exposure to radiation to prevent damage to normal tissue.

14. The therapeutic of claim 12, wherein said therapy is combined with another unrelated therapy.

15. The therapeutic of claim 6, wherein the radiation damage is caused by an event selected from the group consisting of a radiation accident, nuclear accident, nuclear terrorist attack, nuclear war, other radiological emergencies, space travel, radiation therapy, diagnostic radiology, and other similar injuries created by like agents.

16. A model for studying organ specific stem cells, said model comprising an irradiated mouse treated with mesenchymal stems cells capable of secreting extracellular superoxide dismutase for studying the organ specific stem cells.

17. The model of claim 16, wherein said organ specific stem cells are selected from the group consisting of intestinal epithelial stem cells, hematopoietic stem cells, pancreatic endocrine or exocrine stem cells, cardiac muscle stem cells, prostate epithelial stem cells, kidney epithelial stem cells, eye retinal or other eye tissue specific stem cells, lung alveolar stem cells, liver parenchymal stem cells, and neurone specific stem cells.

18. A mesenchymal stem cell genetically altered to secrete extracellular superoxide dismutase for use in therapy.

19. A mesenchymal stem cell genetically altered to secrete extracellular superoxide dismutase for use in treating or preventing radiation damage or other agents having similar mechanisms of action.

20. The use of a mesenchymal stem cell genetically altered to secrete extracellular superoxide dismutase in the preparation of a medicament for treating or preventing radiation damage or other agents having similar mechanisms of action.
